(19) United States
(12) Patent Application Publication
(10) Pub. No.: US 2021/0378968 A1
(43) Pub. Date: Dec. 9, 2021

(54) MULTI-CAVITY CUSTOMIZABLE DOSAGE FORMS

(71) Applicant: Johnson & Johnson Consumer Inc., Skillman, NJ (US)

(72) Inventors: Matthew A. Howard, Zionsville, PA (US); Peyton Hopson, Jacksonville, FL (US); Joshua Sinsel, Quakertown, PA (US); Anthony Bean, Harleysville, PA (US); Vipul Dave, Hillsborough, NJ (US)

(21) Appl. No.: 17/323,392

(22) Filed: May 18, 2021

Related U.S. Application Data
(60) Provisional application No. 63/031,133, filed on May 28, 2020.

(51) Int. Cl. A61K 9/28; A61K 9/20; A61K 45/06

(52) U.S. Cl. CPC A61K 9/28; A61K 9/2095; A61K 45/06; A61K 9/2072; A61K 9/2027; A61K 9/2031; A61K 9/2054

(57) ABSTRACT

An improved customizable dosage form comprising a substrate, such as a tablet core, that has two or more distinct, discrete cavities on opposing sides of its exterior surface; and/or two or more distinct, discrete cavities on a first side of its exterior surface and an identification feature on a second opposing side of its exterior surface. A process for making such a customizable dosage form wherein one or more active ingredients and inactive ingredients such as colors, flavors and/or sensates are deposited into at least one of the cavities.

### CROSS REFERENCE TO RELATED APPLICATIONS

This application claims the benefit of U.S. provisional application 63/031,133 filed on May 28, 2020, the complete disclosure of which is hereby incorporated herein by reference for all purposes.

### FIELD OF THE INVENTION

The present invention relates to a customizable dosage form and a process for making customizable dosage forms wherein one or more active ingredients, colors, flavors and/or sensates are deposited into cavities on the exterior surface of the dosage form.

### BACKGROUND OF THE INVENTION

There is a need in pharmaceutical industry processes to provide dosage forms which can combine high dose active ingredients with low dose active ingredients utilizing various portions of a tablet. Previous processes have often been cumbersome and costly, since they involve preparation of powders in separate unit processes, such as independent granulation steps which are later blended. Previous processes have also been limited to addition of an active ingredient to one portion of a tablet such as in an additional compressed portion, in multilayer tablets, or in an outer coating. This can limit the amount or variety of active ingredients which can be combined.

There also is a need in the pharmaceutical industry to more easily combine ingredients, both active and inactive, into a single dosage form. Powder blends often contain incompatible ingredients in which multiple actives or active and inactive ingredients are in contact within the dosage form. This can lead to undesirable degradation of the active ingredient, especially under accelerated stability conditions.

One solution is additive manufacturing or 3D printing which can be used to create dosage forms that contain several active ingredients, dosage forms with precise amounts of active ingredients and dosage forms that are personalized to an individual patient. Acosta-Velez, G. F. & Wu, B. M., 3D Pharming: Directing Printing of Personalized Pharmaceutical Tablets, Polymer Sciences 2016, 1:2 provides an overview of the manufacture of pharmaceutical tablets through inkjet 3D printing and fused deposition modeling. Trivedi, M. et al., Additive manufacturing of pharmaceuticals for precision medicine applications: A review of the promises and perils in implementation, 23 Additive Manufacturing (2018), 319-28 provides a review of different additive manufacturing processes available for use in pharmaceutical dosage form manufacture and discloses existing dosage form designs specific to additive manufacturing, including multilayer and capsule-in-capsule designs. Wang, L., in The Pharmaceutical Journal (2013, Jul. 3); Printing Medicines, a new era of dispensing and drug formulation; provides a review of 3D printing for medicine manufacturing and personalization. Boehm et al, in Materials Today (2014, volume 17, issue 5); Inkjet Printing for Pharmaceutical Applications; provides a review of printing for miconazole on two dimensional and three dimensional structures. Sandler et al, in Journal of Pharmaceutical Sciences (2011, Volume 100, Issue 8); Inkjet Printing of Drug Substances and use of porous substrates-towards individualized dosing; provides a review depositing of active ingredients onto paper substrates. An article, “Future pills will be personalized and 3 D Printed, just for you”, published in World Changing Ideas (Feb. 1, 2019), describes the first research trial using 3D Printed tablets for children to eventually develop tailored drug dosage in easy-to-take-tablets. Recently, FabRx Ltd., a spin-out from University College London (UCL), released the first pharmaceutical 3D printer M3DIMAKER™ for the manufacture of personalized medicines. Awad et. al, in Drug Discovery Today (Volume 23, Number 8, August 2018), Reshaping drug development using 3D printing, gives an overview of 3D printing techniques in various stages of drug development. Trenfield et al, in the International Journal of Pharmaceutics (Vol 548, 2018), 3D printed drug products: Non destructive dose verification using a rapid point and shoot approach, uses spectroscopy to analyze drug products as part of a 3D printing process. Martinez et. al, in AAPS Pharm Tech (vol 19, No. 8, 2018), Influence of Geometry on the Drug Release Profiles of Sterolithographic (SLA) 3D-Printed tablets evaluates the influence of tablet shape for 3D printed tablets on release rates. Awad et. al, in the International Journal of Pharmaceutics, (vol 548, 2018), 3D printed medicines: A new branch of digital healthcare, reviews 3D printed drug products as part of personalized medicine. Drawbacks for these printing technologies include cost and lack of scalability and speed.

### SUMMARY OF THE INVENTION

The present invention provides an improved dosage form and process to deposit active and inactive ingredients on to such dosage forms. Particularly, the present invention provides a customizable dosage form comprising a substrate, such as a tablet core, that has two or more distinct, discrete cavities on opposing sides of its exterior surface. The present invention provides a customizable dosage from comprising a substrate, such as a tablet core, that has two or more distinct, discrete cavities on one side and an alignment feature on the opposing side. The present invention may also include an identification feature in addition to the alignment feature. Use of such an improved dosage form provides an advantage including improved speed of manufacture and the ability to manufacture dosage forms with different active and inactive ingredient combinations quickly.

The present invention also provides a process for making such a customizable dosage form wherein one or more active ingredients and inactive ingredients such as colors, flavors and/or sensates are deposited into at least one of the cavities. The present invention provides similar benefits as blended or granulated active ingredients, while providing the ability to vary and separate active ingredients from each other and active ingredients from inactive ingredients.

The present invention allows for deposition of active or inactive ingredients in two or more cavities in multiple regions across a tablet. Use of the process to deposit ingredients with the invention provides advantages, including but not limited to, permitting the addition of actives, colors, flavors, sensates and textures; separating incompatible active and inactive ingredients; allowing for customization of dosage forms; providing a perception of speed; permitting taste masking; and providing for visual recognition to aid in product selection.

The compositions that are deposited into individual cavities may have different release rates, wherein one cavity releases an ingredient in the oral cavity for buccal absorption and another cavity releases in a portion of the gastrointestinal tract; whether it be the stomach, duodenum, small intestine, or colon. The gastrointestinal release may be formulated through the addition of an enteric polymer or swellable polymer in the deposition composition.

The invention also allows for multiple and/or layered depositions into a cavity. The dosage form may have multiple deposits in a single cavity and the multiple deposits may have different release rates. The first deposit may release in a portion of the gastrointestinal tract and the second deposit may release in the oral cavity.

### BRIEF DESCRIPTION OF THE DRAWINGS

The drawings described herein are for illustrative purposes only of selected examples and are not intended to limit the scope of the present disclosure.

FIG. 1 shows a top view of an embodiment of a dosage form with distinct, discrete cavities on opposing sides of its exterior surface.

FIG. 2 shows a side view of an embodiment of a dosage form with distinct, discrete cavities on opposing sides of its exterior surface.

FIG. 3 shows an end view of an embodiment of a dosage form with distinct, discrete cavities on opposing sides of its exterior surface.

FIG. 4 shows a side view of an embodiment of a dosage form with distinct, discrete cavities on opposing sides of its exterior surface.

FIG. 5 shows a cross-sectional view of a portion of FIG. 4.

FIG. 6 shows a schematic overview of a process for making dosage forms according to the present invention.

FIG. 7 shows an embodiment of a dosage form with distinct, discrete cavities on opposing sides of its exterior surface.

FIG. 8 shows a top view of an embodiment of a dosage form with distinct, discrete cavities on opposing sides of its exterior surface.

FIG. 9 shows a bottom view of an embodiment of a dosage form with distinct, discrete cavities on opposing sides of its exterior surface.

FIG. 10 shows an end view of an embodiment of a dosage form with distinct, discrete cavities on opposing sides of its exterior surface.

FIG. 11 shows an end view of an embodiment of a dosage form with distinct, discrete cavities on opposing sides of its exterior surface.

FIG. 12 shows a side view of an embodiment of a dosage form with distinct, discrete cavities on opposing sides of its exterior surface.

FIG. 13 shows a side view of an embodiment of a dosage form with distinct, discrete cavities on opposing sides of its exterior surface.

FIG. 14 shows an embodiment of a dosage form with distinct, discrete cavities on opposing sides of its exterior surface.

FIG. 15 shows a top view of an embodiment of a dosage form with distinct, discrete cavities on opposing sides of its exterior surface.

FIG. 16 shows a bottom view of an embodiment of a dosage form with distinct, discrete cavities on opposing sides of its exterior surface.

FIG. 17 shows an end view of an embodiment of a dosage form with distinct, discrete cavities on opposing sides of its exterior surface.

FIG. 18 shows an end view of an embodiment of a dosage form with distinct, discrete cavities on opposing sides of its exterior surface.

FIG. 19 shows a side view of an embodiment of a dosage form with distinct, discrete cavities on opposing sides of its exterior surface.

FIG. 20 shows a side view of an embodiment of a dosage form with distinct, discrete cavities on opposing sides of its exterior surface.

FIG. 21 shows an embodiment of a dosage form with distinct, discrete cavities on opposing sides of its exterior surface.

FIG. 22 shows a top view of an embodiment of a dosage form with distinct, discrete cavities on opposing sides of its exterior surface.

FIG. 23 shows a bottom view of an embodiment of a dosage form with distinct, discrete cavities on opposing sides of its exterior surface.

FIG. 24 shows an end view of an embodiment of a dosage form with distinct, discrete cavities on opposing sides of its exterior surface.

FIG. 25 shows an end view of an embodiment of a dosage form with distinct, discrete cavities on opposing sides of its exterior surface.

FIG. 26 shows a side view of an embodiment of a dosage form with distinct, discrete cavities on opposing sides of its exterior surface.

FIG. 27 shows a side view of an embodiment of a dosage form with distinct, discrete cavities on opposing sides of its exterior surface.

FIG. 28 shows an embodiment of a dosage form with distinct, discrete cavities on one side and an alignment feature on the opposing side.

FIG. 29 shows a top view of an embodiment of a dosage form with distinct, discrete cavities on one side and an alignment feature on the opposing side.

FIG. 30 shows a bottom view of an embodiment of a dosage form with distinct, discrete cavities on one side and an alignment feature on the opposing side.

FIG. 31 shows an end view of an embodiment of a dosage form with distinct, discrete cavities on one side and an alignment feature on the opposing side.

FIG. 32 shows an end view of an embodiment of a dosage form with distinct, discrete cavities on one side and an alignment feature on the opposing side.

FIG. 33 shows a side view of an embodiment of a dosage form with distinct, discrete cavities on one side and an alignment feature on the opposing side.

FIG. 34 shows a side view of an embodiment of a dosage form with distinct, discrete cavities on one side and an alignment feature on the opposing side.

FIG. 35 shows an embodiment of a dosage form with distinct, discrete cavities on one side and an alignment feature on the opposing side.

FIG. 36 shows a top view of an embodiment of a dosage form with distinct, discrete cavities on one side and an alignment feature on the opposing side.

FIG. 37 shows a bottom view of an embodiment of a dosage form with distinct, discrete cavities on one side and an alignment feature on the opposing side.

FIG. 38 shows an end view of an embodiment of a dosage form with distinct, discrete cavities on one side and an alignment feature on the opposing side.

FIG. 39 shows an end view of an embodiment of a dosage form with distinct, discrete cavities on one side and an alignment feature on the opposing side.

FIG. 40 shows a side view of an embodiment of a dosage form with distinct, discrete cavities on one side and an alignment feature on the opposing side.

FIG. 41 shows a side view of an embodiment of a dosage form with distinct, discrete cavities on one side and an alignment feature on the opposing side.

FIGS. 42-50 show a bottom view of an embodiment of a dosage form with distinct, discrete cavities on one side and an alignment feature on the opposing side.

FIG. 51 shows an embodiment of a dosage form with distinct, discrete cavities on one side and an alignment feature and identification feature on the opposing side.

FIG. 52 shows a top view of an embodiment of a dosage form with distinct, discrete cavities on one side and an alignment feature and identification feature on the opposing side.

FIG. 53 shows a bottom view of an embodiment of a dosage form with distinct, discrete cavities on one side and an alignment feature and identification feature on the opposing side.

FIG. 54 shows an end view of an embodiment of a dosage form with distinct, discrete cavities on one side and an alignment feature and identification feature on the opposing side.

FIG. 55 shows an end view of an embodiment of a dosage form with distinct, discrete cavities on one side and an alignment feature and identification feature on the opposing side.

FIG. 56 shows a side view of an embodiment of a dosage form with distinct, discrete cavities on one side and an alignment feature and identification feature on the opposing side.

FIG. 57 shows a side view of an embodiment of a dosage form with distinct, discrete cavities on one side and an alignment feature and identification feature on the opposing side.

FIG. 58 shows an embodiment of a dosage form with distinct, discrete cavities on one side and an alignment feature and identification feature on the opposing side.

FIG. 59 shows a top view of an embodiment of a dosage form with distinct, discrete cavities on one side and an alignment feature and identification feature on the opposing side.

FIG. 60 shows a bottom view of an embodiment of a dosage form with distinct, discrete cavities on one side and an alignment feature and identification feature on the opposing side.

FIG. 61 shows an end view of an embodiment of a dosage form with distinct, discrete cavities on one side and an alignment feature and identification feature on the opposing side.

FIG. 62 shows an end view of an embodiment of a dosage form with distinct, discrete cavities on one side and an alignment feature and identification feature on the opposing side.

FIG. 63 shows a side view of an embodiment of a dosage form with distinct, discrete cavities on one side and an alignment feature and identification feature on the opposing side.

FIG. 64 shows a side view of an embodiment of a dosage form with distinct, discrete cavities on one side and an alignment feature and identification feature on the opposing side.

### DETAILED DESCRIPTION OF INVENTION

As used herein, the term “dosage form” applies to any solid composition designed to contain a specific pre-determined amount (dose) of a certain ingredient, for example an active ingredient as defined below. Suitable dosage forms may be pharmaceutical drug delivery systems, including those for oral administration, buccal administration, rectal administration, topical or mucosal delivery, or subcutaneous implants, or other implanted drug delivery systems; or compositions for delivering minerals, vitamins and other nutraceuticals, oral care agents, flavorants, and the like. The dosage form may be an orally administered system for delivering a pharmaceutical active ingredient to the gastro-intestinal tract of a human. The dosage form may also be an orally administered “placebo” system containing pharmaceutically inactive ingredients, and the dosage form is designed to have the same appearance as a particular pharmaceutically active dosage form, such as may be used for control purposes in clinical studies to test, for example, the safety and efficacy of a particular pharmaceutically active ingredient.

### Tablet, Core and Cavity Definition

As used herein the term “tablet” refers to a solid form prepared by compaction of powders on a tablet press, as well known in the pharmaceutical arts. Tablets can be made in a variety of shapes, including round, or elongated, such as flattened ovoid or cylindrical shapes. Tablets may also have a tapered and/or notched end to allow for consistent physical orientation of the tablet during manufacturing.

The core (or substrate) may be any solid form. The core may be prepared by any suitable method, for example the core be a compressed dosage form, or may be molded. As used herein, “substrate” refers to a surface or underlying support, upon which another substance resides or acts, and “core” refers to a material that is at least partially in contact with a portion of another material or surrounded by another material. For the purposes of the present invention, the terms may be used interchangeably: i.e., the term “core” may also be used to refer to a “substrate.” Preferably, the core comprises a solid, for example, the core may be a compressed or molded tablet, hard or soft capsule, suppository, or a confectionery form such as a lozenge, nougat, caramel, fondant, or fat based composition.

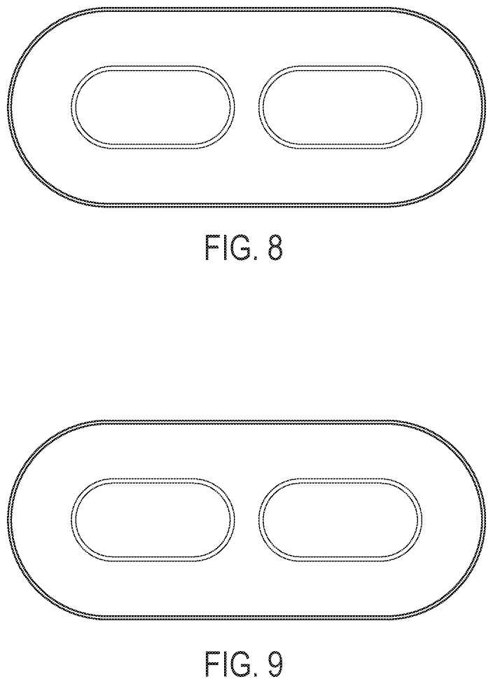

The core may have one or more major faces. The core may be in a variety of different shapes. For example, the core may be in the shape of a truncated cone. In other examples, the core may be shaped as a polyhedron, such as a cube, pyramid, prism, or the like; or may have the geometry of a space figure with some non-flat faces, such as a cone, cylinder, or the like. Exemplary core shapes that may be employed include tablet shapes formed from compression tooling shapes described by “The Elizabeth Companies Tablet Design Training Manual” (Elizabeth Carbide Die Co., Inc., p. 7 (McKeesport, Pa.) (incorporated herein by reference) as follows (the tablet shape corresponds inversely to the shape of the compression tooling):
Shallow Concave.
Standard Concave.
Deep Concave.
Extra Deep Concave.
Modified Ball Concave.
Standard Concave Bisect.
Standard Concave Double Bisect.
Standard Concave European Bisect.
Standard Concave Partial Bisect.
Double Radius.
Bevel & Concave.
Flat Plain.
Flat-Faced-Beveled Edge (F.F.B.E.).
F.F.B.E. Bisect.
F.F.B.E. Double Bisect.
Ellipse.
Oval.
Capsule.
Rectangle.
Pentagon.
Octagon.
Diamond.
Arrowhead.
Bullet.
Barrel.
Half Moon.
Shield.
Heart.
Almond.
Parallelogram.
Trapezoid.
FIG. 8/Bar Bell.
Bow Tie.
Uneven Triangle.

The core may be pressed of a blend of suitable active ingredients and excipients which may be either their natural color, including white, or can be conventionally colored as desired to provide a core of any desired color.

As used herein the term cavity refers to a recess in the surface or face of a substrate or core designed to receive a deposited portion. The deposited portion may or may not contain an active ingredient.

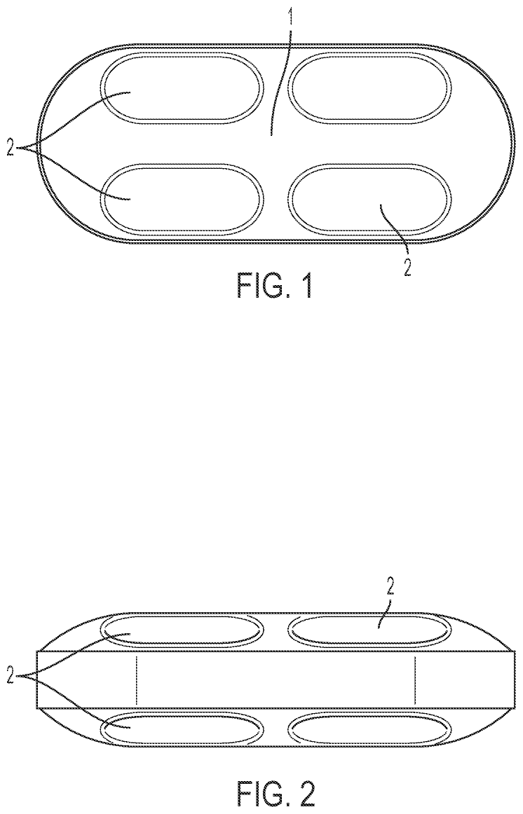

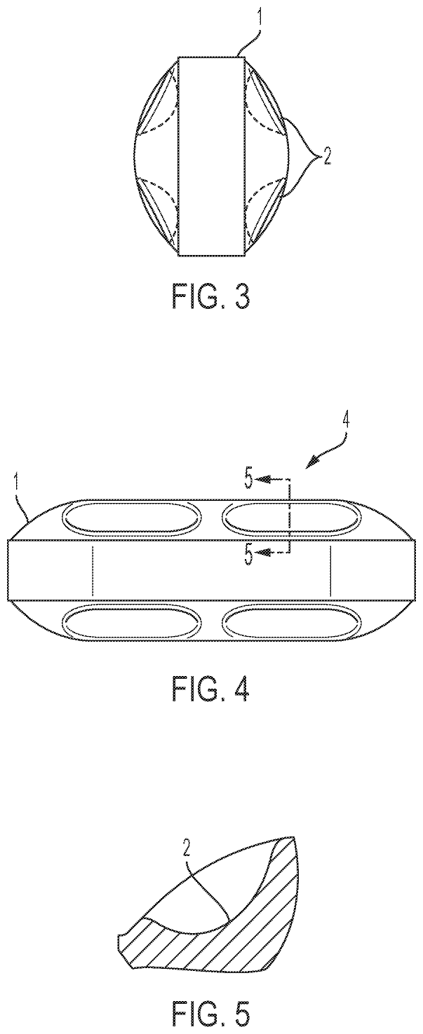

The core of the present invention may comprise multiple cavities on multiple faces of the tablet, including opposing surfaces of the tablet. As shown in FIGS. 1-3, cavities 2 are present on opposing surfaces of the tablet 1.

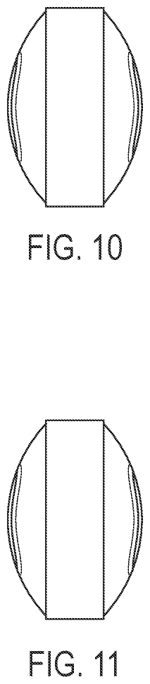

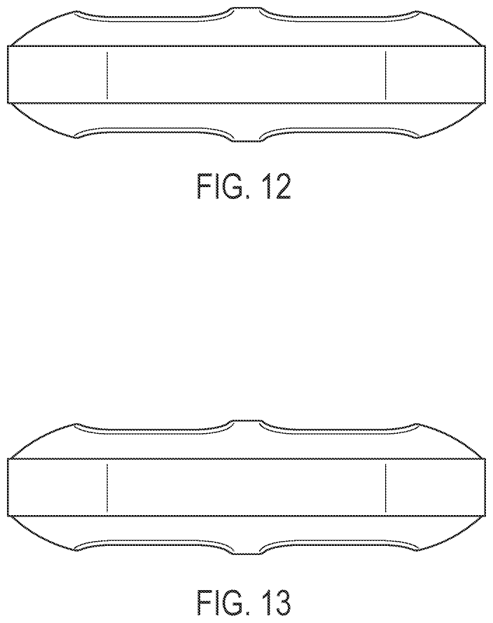

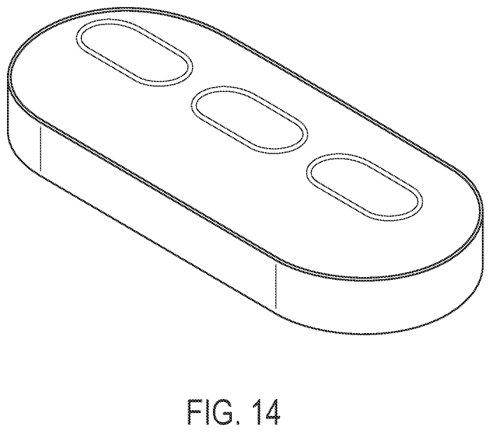

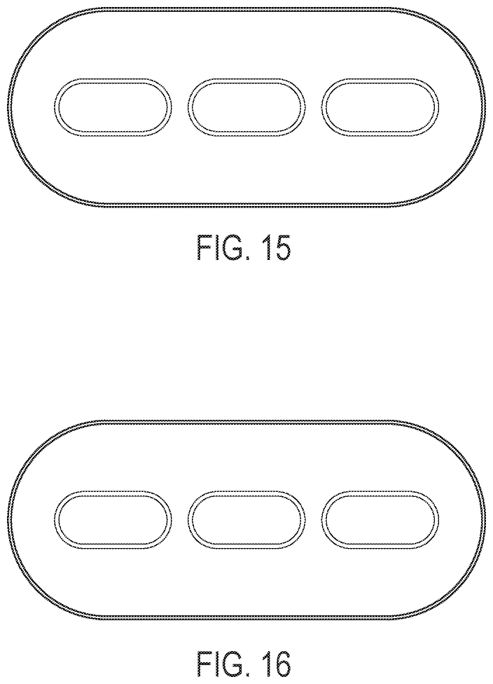

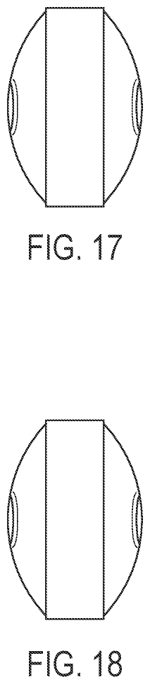

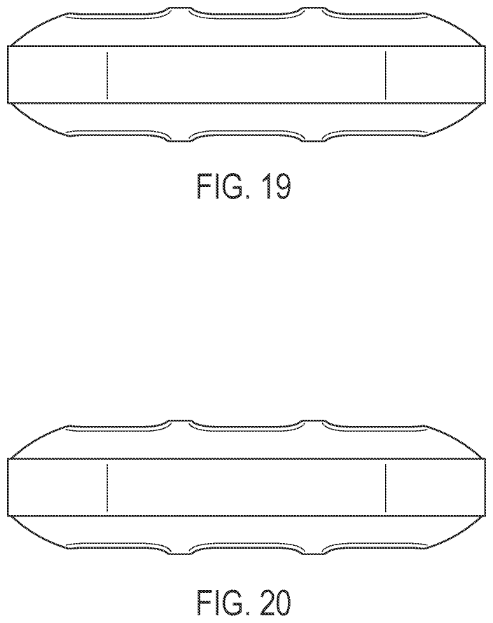

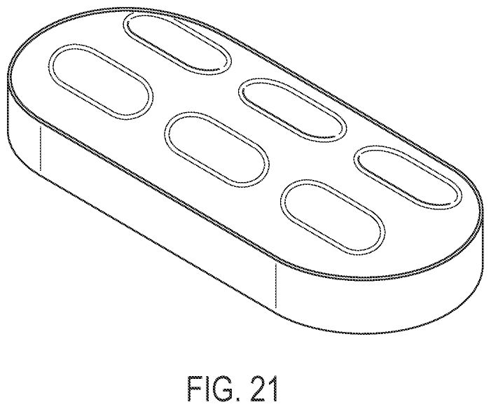

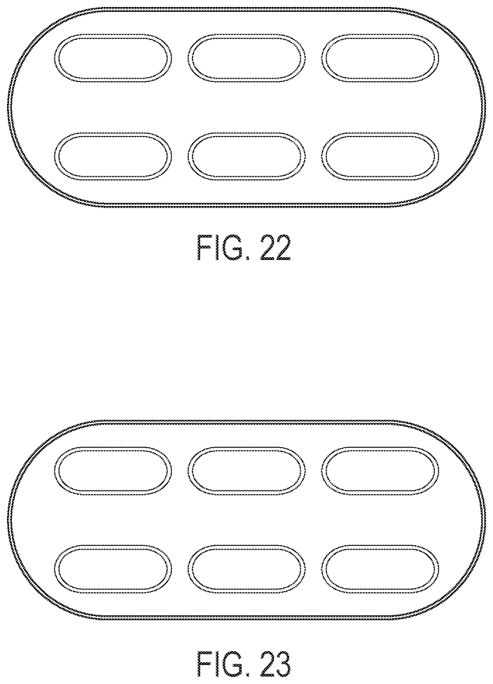

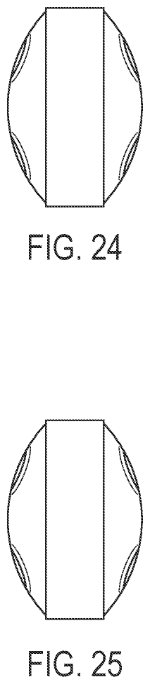

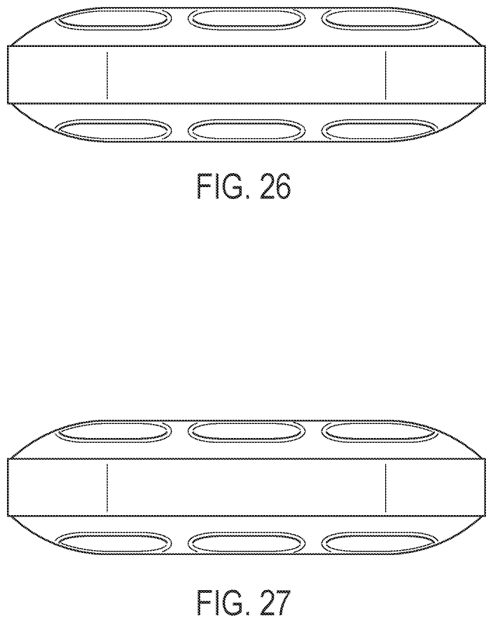

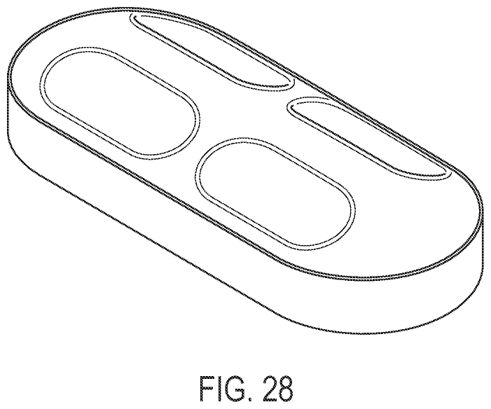

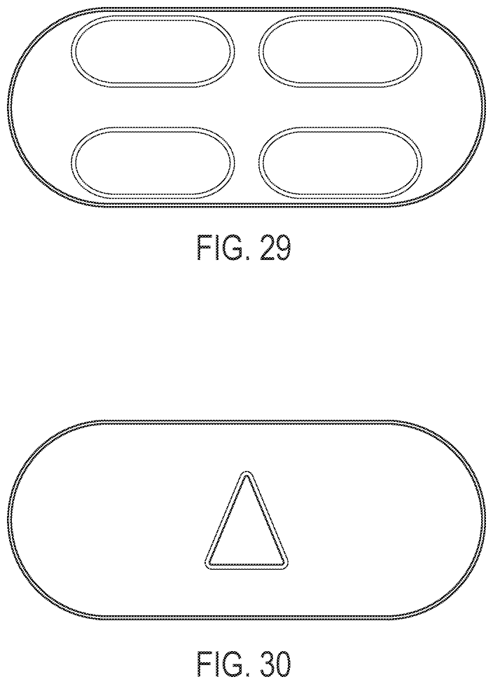

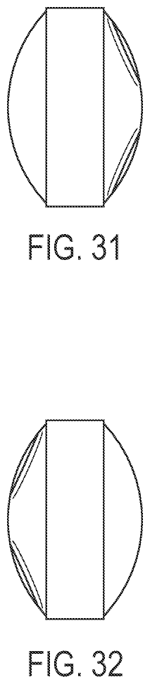

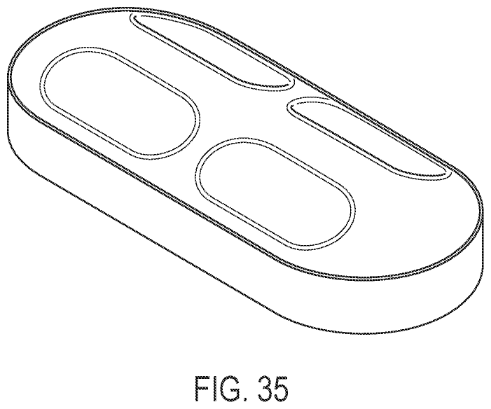

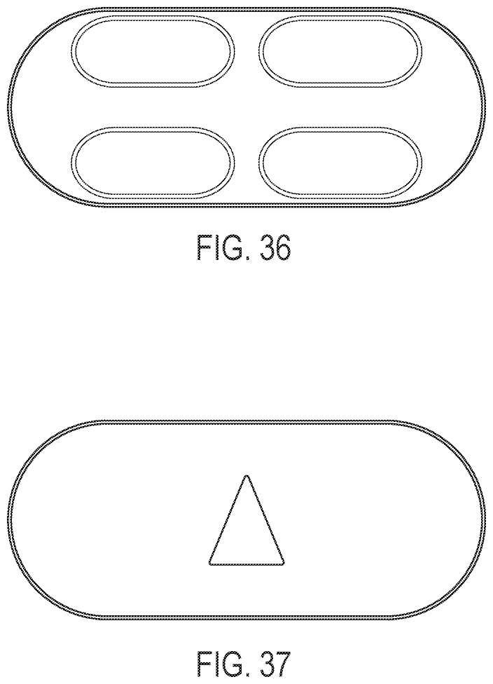

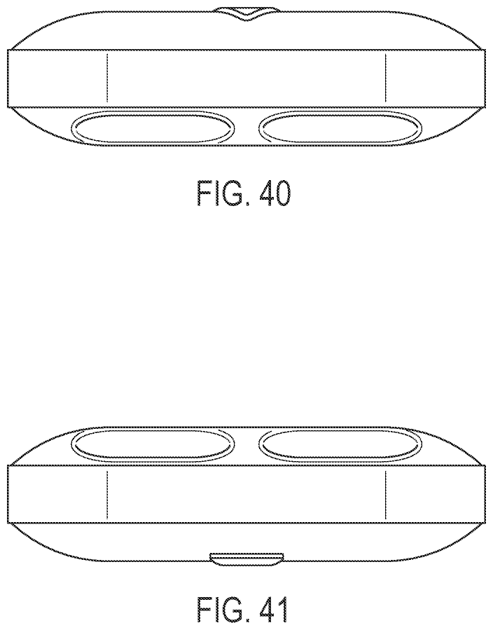

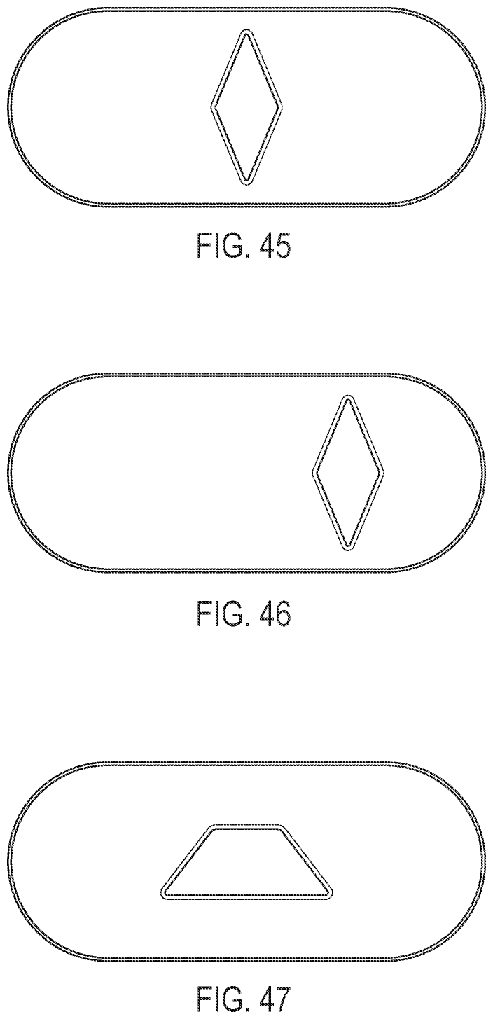

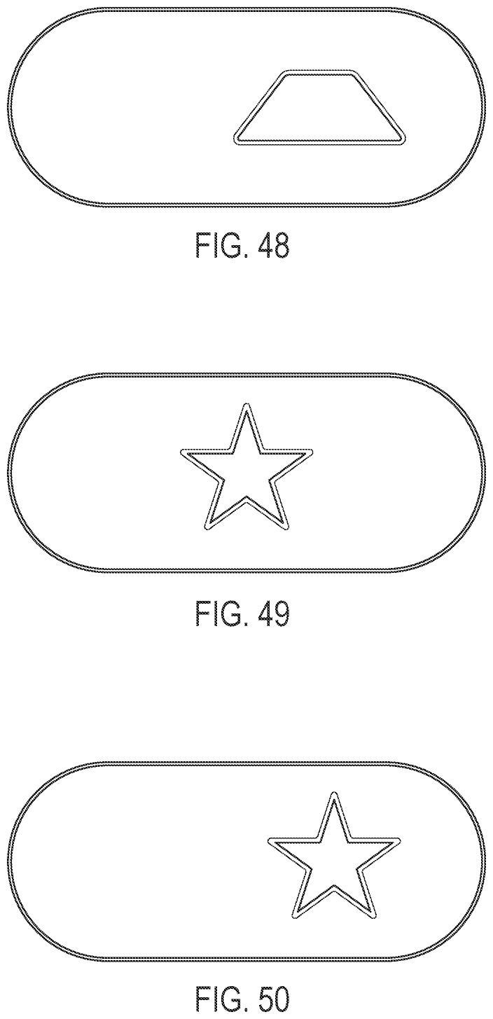

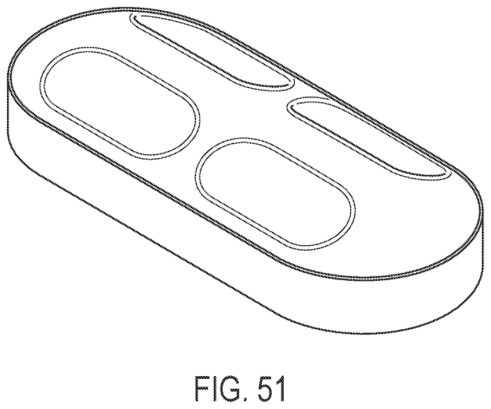

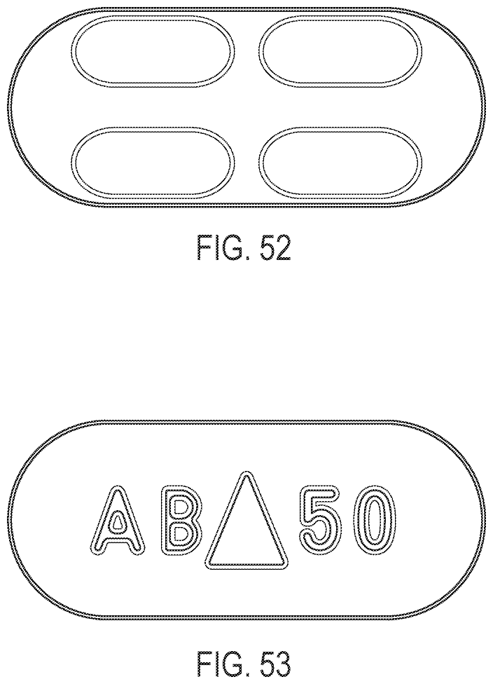

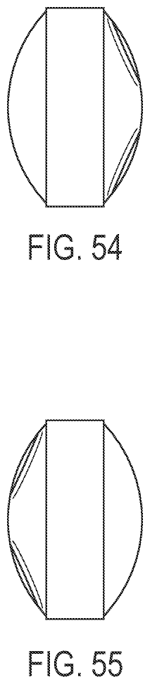

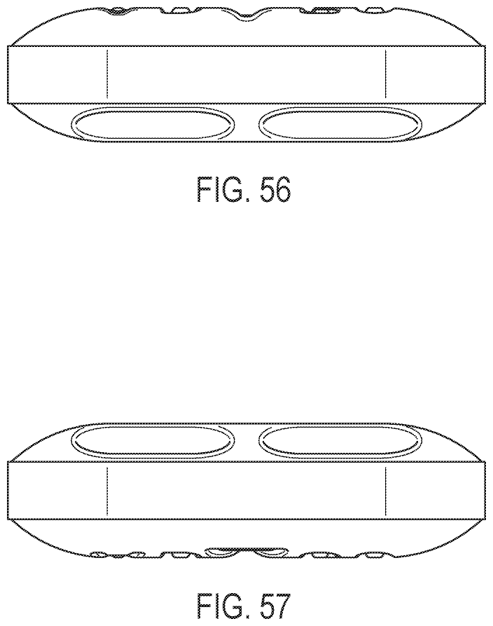

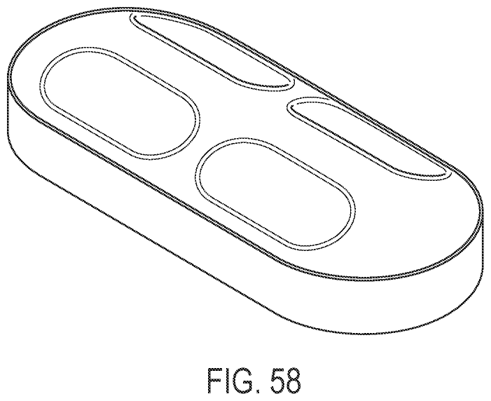

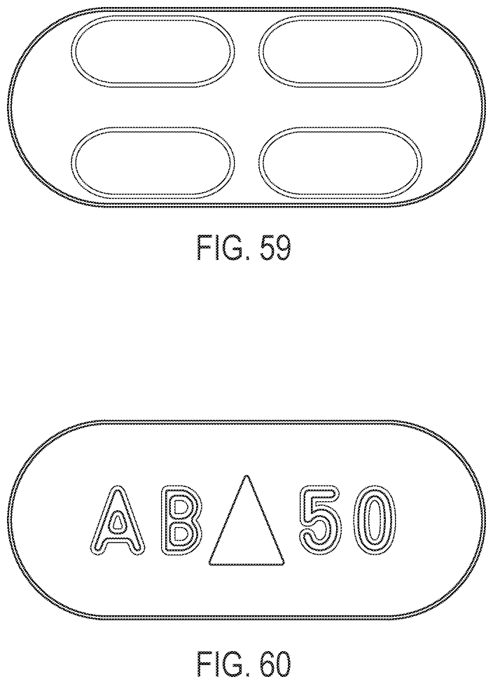

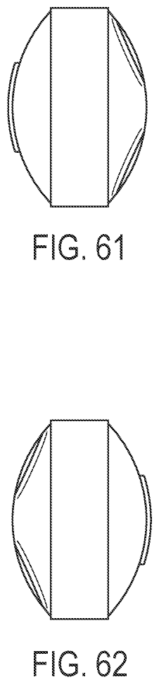

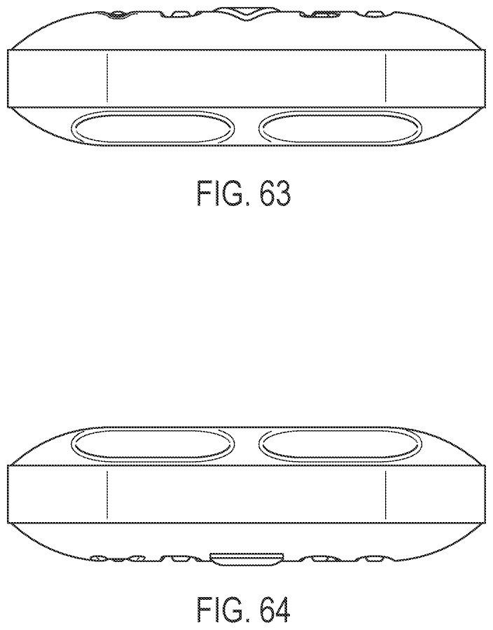

The core may have any number and size of cavities on one or both surfaces of the tablet. The core may have up to 12 cavities on the tablet, including from about 2 to about 6 cavities on one surface of the tablet and from about 2 to about 6 cavities on the second surface of the tablet, including 6 cavities on one surface of the tablet and 6 cavities on the second surface of the tablet, 5 cavities on one surface of the tablet and 5 cavities on the second surface of the tablet, 4 cavities on one surface of the tablet and 4 cavities on the second surface of the tablet, 3 cavities on one surface of the tablet and 3 cavities on the second surface of the tablet, and 2 cavities on one surface of the tablet and 2 cavities on the second surface of the tablet. The core may also have up to 8 cavities on the tablet, including from about 2 to about 4 cavities on one surface of the tablet and from about 2 to about 4 cavities on the second surface of the tablet. The core may also have up to 6 cavities on the tablet, including 2 cavities on one surface of the tablet, 3 cavities on one surface of the tablet, 4 cavities on one surface of the tablet, 5 cavities on one surface of the tablet, and 6 cavities on one surface of the tablet. As shown in FIGS. 1-5, the core may have 4 cavities on one surface of the tablet and 4 cavities on an opposing surface of the tablet. As shown in FIGS. 7-13, the core may have 2 cavities on one surface of the tablet and 2 cavities on an opposing surface of the tablet. As shown in FIGS. 14-20, the core may have 3 cavities on one surface of the tablet and 3 cavities on an opposing surface of the tablet. As shown in FIGS. 21-27, the core may have 6 cavities on one surface of the tablet and 6 cavities on an opposing surface of the tablet. As shown in FIGS. 29-64, the core may have cavities on only one surface of the tablet.

The cavities on the core may be positioned so that they are in the same orientation or shape of the tablet. If the core is an elongated tablet shape, the cavities may also be elongated.

The cavities on the core may be physically separated by a portion of the surface of the core, with a portion of the surface between each cavity at least 1 mm, or at least 2 mm.

The term “deposition” and “deposited portion” refers to the placement and/or dispensing of a flowable material and said portion into a cavity of a dosage form, from a repository of said flowable material.

As shown in FIGS. 4 and 5, the cavities may be shaped such that they contour with the surface of the dosage form. FIG. 5 depicts the cross-section of B-B 4 in FIG. 4. Here, the contoured cavities allow the flowable material to spread evenly across the cavity as a function of area and uniformly within the cavity as a function of depth during deposition.

### Alignment Feature and Identification Feature

As used herein the term alignment feature refers to a feature that is designed to orient a tablet during the manufacturing process. Alignment features include a recess in the surface or face of a substrate or core, a protrusion in the surface or face of a substrate or core and a symbol, marker or other visual cue printed onto the surface or face of a substrate or core. Alignment features also include a tapered and/or notched end of a dosage form.

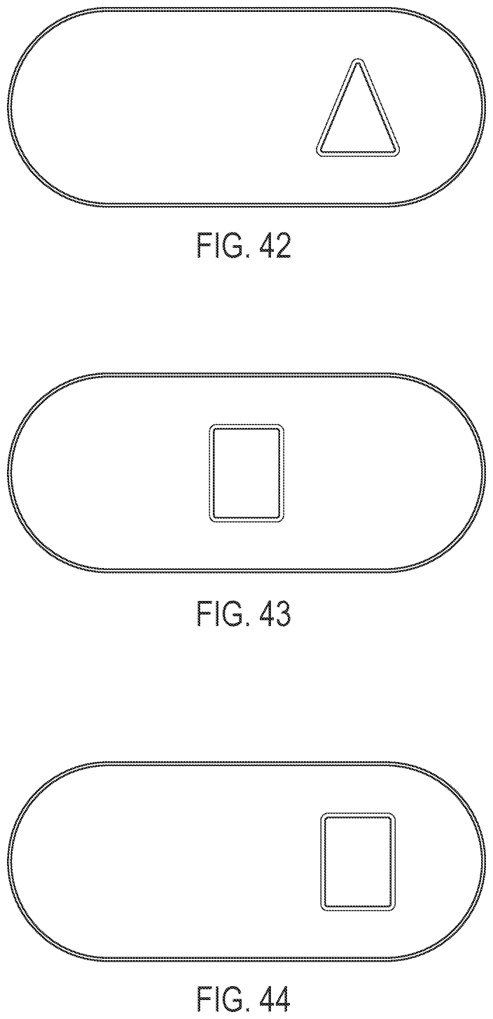

The alignment feature may be any shape that allows for consistent seating or orientation of the tablet throughout the manufacturing process. The alignment feature may be a triangle, rectangle, elongated diamond, trapezoid or star. FIGS. 37 and 42-50 depict examples of alignment features. The alignment feature may be placed at the center of the tablet surface as shown in FIGS. 37, 43, 45, 47 and 49. The alignment feature may be placed off-center on the tablet surface as shown in FIGS. 42, 44, 46, 48 and 50. The alignment feature may be placed at or near the edge of the tablet surface.

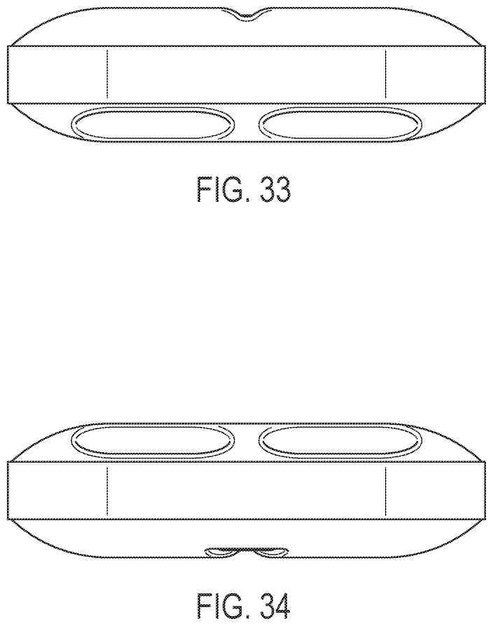

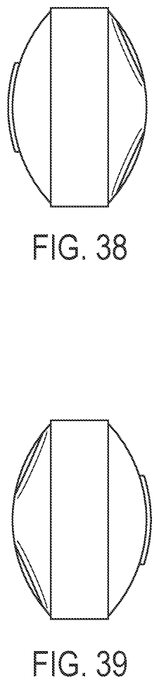

The alignment feature may be a recess or hollow portion of the tablet surface that is designed to receive or be seated in a corresponding shape. FIGS. 33-34 and 56-57 depict examples of a recessed alignment feature. The alignment feature may be a protrusion or raised portion of the tablet surface that is designed to be inserted into a corresponding recess or hollow. FIGS. 38-41 and 61-64 depict examples of a protruding alignment feature.

The alignment feature may be a symbol, cue or marker stamped or printed onto the surface or face of a substrate or core. The symbol, cue or marker may contain ink, colorant, metal or other chemical which can be identified by a video, visual or spectroscopic system. Such video, visual or spectroscopic systems may track the symbol, cue or marker in order to ensure the correct physical orientation of the dosage form for deposition.

The alignment feature may allow for better precision, accuracy and speed during deposition of ingredients into the cavities.

As used herein the term identification feature refers to any markings, letters or numbers or combinations thereof that provide information to a consumer about the dosage form. Such information may include active ingredient, amount of active ingredient, manufacturer and/or brand name. FIGS. 53 and 60 provide examples of identification features.

### Core Ingredients

The core may contain a disintegrant and/or a superdisintegrant. Suitable disintegrants for making the core, or a portion thereof, by compression, include, e.g., sodium starch glycolate, cross-linked polyvinylpyrrolidone, cross-linked carboxymethylcellulose, starches, microcrystalline cellulose, and the like. The superdisintegrant may be present as a percentage of the weight of the core from about 0.05 percent to about 10 percent.

The dosage form of the present invention preferably contains one or more active ingredients. Suitable active ingredients broadly include, for example, pharmaceuticals, minerals, vitamins and other nutraceuticals, oral care agents, flavorants and mixtures thereof. Suitable pharmaceuticals include analgesics, anti-inflammatory agents, antiarthritics, anesthetics, antihistamines, anti-smoking agents, antitussives, antibiotics, anti-infective agents, antivirals, anticoagulants, antidepressants, antidiabetic agents, antiemetics, antiflatulents, antifungals, antispasmodics, appetite suppressants, bronchodilators, cardiovascular agents, central nervous system agents, central nervous system stimulants, decongestants, oral contraceptives, diuretics, expectorants, gastrointestinal agents, migraine preparations, motion sickness products, mucolytics, muscle relaxants, oncology agents, osteoporosis preparations, polydimethylsiloxanes, respiratory agents, sleep-aids, urinary tract agents and mixtures thereof.

Suitable flavorants include menthol, peppermint, mint flavors, fruit flavors, chocolate, vanilla, bubblegum flavors, coffee flavors, liqueur flavors and combinations and the like.

Examples of suitable gastrointestinal agents include antacids such as calcium carbonate, magnesium hydroxide, magnesium oxide, magnesium carbonate, aluminum hydroxide, sodium bicarbonate, dihydroxyaluminum sodium carbonate; stimulant laxatives, such as bisacodyl, cascara sagrada, danthron, senna, phenolphthalein, aloe, castor oil, ricinoleic acid, and dehydrocholic acid, and mixtures thereof; H2 receptor antagonists, such as famotidine, ranitidine, cimetadine, nizatidine; proton pump inhibitors such as omeprazole or lansoprazole; gastrointestinal cytoprotectives, such as sucraflate and misoprostol; gastrointestinal prokinetics, such as prucalopride, antibiotics for H. pylori, such as clarithromycin, amoxicillin, tetracycline, and metronidazole; antidiarrheals, such as diphenoxylate, loperamide and racecadotril; glycopyrrolate; antiemetics, such as ondansetron, analgesics, such as mesalamine.

At least one active ingredient may be selected from bisacodyl, famotidine, ranitidine, cimetidine, prucalopride, diphenoxylate, loperamide, lactase, mesalamine, bismuth, antacids, and pharmaceutically acceptable salts, esters, isomers, and mixtures thereof.

At least one active ingredient may be selected from analgesics, anti-inflammatories, and antipyretics, e.g., non-steroidal anti-inflammatory drugs (NSAIDs), including a) propionic acid derivatives, e.g., ibuprofen, naproxen, ketoprofen and the like; b) acetic acid derivatives, e.g., indomethacin, diclofenac, sulindac, tolmetin, and the like; c) fenamic acid derivatives, e.g., mefenamic acid, meclofenamic acid, flufenamic acid, and the like; d) biphenylcarbodylic acid derivatives, e.g., diflunisal, flufenisal, and the like; e) oxicams, e.g., piroxicam, sudoxicam, isoxicam, meloxicam, and the like; f) cyclooxygenase-2 (COX-2) selective NSAIDs; and g) pharmaceutically acceptable salts of the foregoing.

The active ingredient or ingredients are present in the dosage form in a therapeutically effective amount, which is an amount that produces the desired therapeutic response upon oral administration and can be readily determined by one skilled in the art. In determining such amounts, the particular active ingredient being administered, the bioavailability characteristics of the active ingredient, the dosing regimen, the age and weight of the patient, and other factors should be considered, as known in the art. Typically, the dosage form comprises at least about 1 weight percent, preferably, the dosage form comprises at least about 5 weight percent, e.g., about 20 weight percent of one or more active ingredients. The core may comprise a total of at least about 25 weight percent (based on the weight of the core) of one or more active ingredients.

The active ingredient or ingredients may be present in the dosage form in any form. For example, one or more active ingredients may be dispersed at the molecular level, e.g., melted or dissolved, within the dosage form, or may be in the form of particles, which in turn may be coated or uncoated. If an active ingredient is in the form of particles, the particles (whether coated or uncoated) typically have an average particle size of about 1-2000 microns. Such particles may be crystals having an average particle size of about 1300 microns. The particles may also be granules or pellets having an average particle size of about 50-2000 microns, preferably about 50-1000 microns, most preferably about 100-800 microns.

The dissolution characteristics of the at least one active ingredient may follow an “immediate release profile”. As used herein, an immediate release profile is one in which the active ingredient dissolves without substantial delay or retardation due to the dosage form. This can be contrasted with the dissolution of modified release, e.g., delayed or controlled release dosage forms known in the art. The dissolution rate of the immediately released active ingredient from the dosage form of the invention may be within about 20% of the dissolution rate of the active ingredient from a pure crystalline powder of said active ingredient, e.g., the time for 50%, 75%, 80%, or 90% dissolution of active ingredient from the dosage form is not more than 20% longer than the corresponding time for 50%, 75%, 80%, or 90% dissolution of active ingredient from a pure crystalline powder of said active ingredient. The dissolution of the immediately released active ingredient from the dosage form may also meet USP specifications for immediate release tablets, gelcaps, or capsules containing the active ingredient. For example, for acetaminophen tablets, USP 24 specifies that in pH 5.8 phosphate buffer, using USP apparatus 2 (paddles) at 50 rpm, at least 80% of the acetaminophen contained in the dosage form is released therefrom within 30 minutes after dosing; and for acetaminophen and codeine phosphate capsules USP 24 specifies that at least 75% of the acetaminophen contained in the dosage form is dissolved within 30 minutes in 900 mL of 0.1 N Hydrochloric acid using USP Apparatus 2 (paddles) at 50 rpm; and for ibuprofen tablets, USP 24 specifies that in pH 7.2 phosphate buffer, using USP apparatus 2 (paddles) at 50 rpm, at least 80% of the ibuprofen contained in the dosage form is released therefrom within 60 minutes. See USP 24, 2000 Version, 19-20 and 856 (1999). The immediately released active ingredient may be acetaminophen, and when tested in 37° C. water using USP Apparatus II (paddles) at 50 rpm, at least 80%, preferably at least 85%, of the acetaminophen contained in the dosage form is released therefrom within 30 minutes.

The time for release of at least 80%, preferably at least 85%, of at least one active ingredient contained in the dosage form is released therefrom may not be more than about 50%, e.g., not more than about 40% of the time specified by the dissolution method for immediate release listed in the United States New Drug Application for that particular active ingredient.

When the immediately released active ingredient is acetaminophen, when tested in 37° C. water using USP Apparatus II (paddles) at 50 rpm, at least 80% of the acetaminophen contained in the dosage form is released therefrom within about 6 minutes, e.g., within about 5 minutes, or within about 3 minutes.

The tablet and deposited portions can be observed using the USP Disintegration test as outlined in USP 34-NF29, Section 701. The tablet and coating positions can also be observed by placing the tablet into water at 37° C. without agitation.

Disintegration of the tablet without agitation can be observed at less than about 30 seconds, e.g., less than about 15 seconds, e.g., less than about 10 seconds, e.g., less than about 5 seconds.

At least one active ingredient may be selected from propionic acid derivative NSAID, which are pharmaceutically acceptable analgesics/non-steroidal anti-inflammatory drugs having a free —CH(CH₃)COOH or —CH₂CH₂COOH or a pharmaceutically acceptable salt group, such as —CH(CH₃)COO-Na+ or CH₂CH₂COO-Na+, which are typically attached directly or via a carbonyl functionality to a ring system, preferably an aromatic ring system.

Examples of useful propionic acid derivatives include ibuprofen, naproxen, benoxaprofen, naproxen sodium, fenbufen, flurbiprofen, fenoprofen, fenbuprofen, ketoprofen, indoprofen, pirprofen, carpofen, oxaprofen, pranoprofen, microprofen, tioxaprofen, suprofen, alminoprofen, tiaprofenic acid, fluprofen, bucloxic acid, and pharmaceutically acceptable salts, derivatives, and combinations thereof. In one embodiment of the invention, the propionic acid derivative is selected from ibuprofen, ketoprofen, flubiprofen, and pharmaceutically acceptable salts and combinations thereof. In another embodiment, the propionic acid derivative is ibuprofen, 2-(4-isobutylphenyl) propionic acid, or a pharmaceutically acceptable salt thereof, such as the arginine, lysine, or histidine salt of ibuprofen. Other pharmaceutically acceptable salts of ibuprofen are described in U.S. Pat. Nos. 4,279,926, 4,873,231, 5,424,075 and 5,510,385, the contents of which are incorporated by reference.

At least one active ingredient may be an analgesic selected from acetaminophen, acetyl salicylic acid, ibuprofen, naproxen, ketoprofen, flurbiprofen, diclofenac, cyclobenzaprine, meloxicam, rofecoxib, celecoxib, metamizol sodic (dypirone), caffeine, and pharmaceutically acceptable salts, esters, isomers, and mixtures thereof.

At least one active ingredient may be selected from phenylephrine, pseudoephedrine, phenylpropanolamine, chlorpheniramine, carbinoxamine, doxylamine, dextromethorphan, diphenhydramine, astemizole, terfenadine, fexofenadine, loratadine, desloratadine, cetirizine, acetylcysteine, guaifenesin, carbocysteine, ambroxol, bromhexine, mixtures thereof and pharmaceutically acceptable salts, esters, isomers, and mixtures thereof.

The at least one active ingredient may be an NSAID and/or acetaminophen, and pharmaceutically acceptable salts thereof.

The core may be covered with a coating that can be any number of medicinally acceptable coverings. The coating may be added prior to or after the cavity deposited portions. The use of coatings is well known in the art and disclosed in, for example, U.S. Pat. No. 5,234,099, which is incorporated by reference herein. Any composition suitable for film-coating a tablet may be used as a coating according to the present invention. Examples of suitable coatings are disclosed in U.S. Pat. Nos. 4,683,256, 4,543,370, 4,643,894, 4,828,841, 4,725,441, 4,802,924, 5,630,871, and 6,274,162, which are all incorporated by reference herein. Suitable compositions for use as coatings include those manufactured by Colorcon, a division of Berwind Pharmaceutical Services, Inc., 415 Moyer Blvd., West Point, Pa. 19486 under the tradename “OPADRY®” (a dry concentrate comprising film forming polymer and optionally plasticizer, colorant, and other useful excipients). Additional suitable coatings include one or more of the following ingredients: cellulose ethers such as hydroxypropylmethylcellulose, hydroxypropylcellulose, and hydroxyethylcellulose; polycarbohydrates such as xanthan gum, starch, and maltodextrin; plasticizers including for example, glycerin, polyethylene glycol, propylene glycol, dibutyl sebecate, triethyl citrate, vegetable oils such as castor oil, surfactants such as Polysorbate-80, sodium lauryl sulfate and dioctyl-sodium sulfosuccinate; polycarbohydrates, pigments, opacifiers.

Preferred coatings include water soluble polymers selected from the group consisting of hydroxypropylmethyl cellulose, hydroxypropyl cellulose, methyl cellulose, polymethacrylates, polyvinyl alcohol, polyvinyl alcohol:polyethylene glycol copolymers and mixtures thereof.

The average thickness of the coating is preferably in the range from about 1 to about 150 microns, or from about 50 to about 90 microns, or from about 10 to about 90 microns, or from about 20 to about 80 microns, or from about 30 to about 70 microns.

The coating may comprise from about 10 percent to about 50 percent, e.g., from about 15 percent to about 20 percent of HPMC. The dried coating typically is present in an amount, based upon the dry weight of the core, from above about 0 percent to about 5 percent, or from about 1 percent to about 4 percent, or from about 2 percent to about 3 percent, or from about 1 to about 2 percent. The coating composition is optionally tinted or colored with colorants such as pigments, dyes and mixtures thereof.

A layer of coating may be applied to the entire exterior surface of core prior to application of the deposited portion. Coating can be applied as a clear, transparent coating such that the core can be seen. The choice is dictated by the preference of the manufacturer and the economics of the product. A commercially available pigment may be included the coating composition in sufficient amounts to provide an opaque film having a visibly distinguishable color relative to the core. The coating may be added after the deposited portion is added to the tablet.

### Deposited Portion

The tablet of the present invention may comprise from about one to about four cavities on one or both major faces. The cavities may comprise a deposited portion. The cavity may be designed to receive a deposit of up to about 50 mg (or 0.05 mL of solution), or up to about 10 mg (or 0.01 mL of solution).

In the present invention, an active ingredient is first incorporated into a flowable form or flowable material so that it can be deposited (as a deposited portion) in the cavity(ies) of the tablet. The flowable form may be a solution, emulsion, gel, suspension, melted solution, melted suspension, or semisolid. The flowable form is subsequently solidified via cooling, drying or a mixture of both to become a final deposited portion.

The deposited portion of the present invention may comprise at least one polymer. In addition, the deposited portion may comprise a surfactant. Suitable surfactants may include nonionic surfactants such as sorbitan esters, polysorbates, or poloxamers.

The tablet comprising deposited portions of the invention provides an observable means of differentiation. The term “observable” (and forms thereof such as “observably,” “observing,” etc.) is intended to have its common meaning, i.e., perceptible (or “perceptibly,” perceiving,” etc. as appropriate) using any one or more of the five human senses, e.g., sight, sound, touch, taste and smell. The tablet comprising deposited portions described herein can employ interaction with one or more of the five senses, and particularly may employ visual, audible and tactile interaction or combinations thereof. Preferably, the tablet comprising deposited portions employs interaction with the visual sense.

The deposited portions of the present invention may comprise at least one active ingredient. The deposited portion may comprise two or more active ingredients. The deposited portion may comprise an inactive ingredient such as a sweetener, a flavor, a color or sensate which is separate and distinct from the sweetener, flavor, color or sensate in another deposited portion on the surface of the tablet. The deposited portion may be substantially free of a pharmaceutical active ingredient and contain an inactive ingredient such as colorant and/or a sweetener, flavor, sensate or mixture thereof.

One measure of the flowable form prior to deposition is viscosity. The viscosity of this flowable form may be from 10 to 2000 centipoise, or from 50 to 500 centipoise, or from 50 centipoise to 300 centipoise when measured using a Brookfield viscometer at 25° C.

The deposited portions of the present invention may be measured for consistency and accuracy in a variety of ways. The deposited portion may be measured as a function of “spread”, wherein the deposited portion spreads to a percentage of the area within the cavity, which is then measured as that which is covered by the deposited portion. The percentage of area in which the deposited portion spreads within at least one cavity may be at least 50 percent, or at least about 60 percent or at least about 70 percent.

In some cases the cavity spread has the undesirable effect of being greater than 100 percent of the area of the cavity. This may occur with uncoated tablets. In some cases the cavity spread is between 70 percent and 100 percent with a tablet comprising at least 0.1 percent of a film coating by weight of the core.

Another measure of the deposited portion is that of diffusion, or the level or length at which the deposited portion diffuses into the body of the tablet. This can be measured by adding a colorant to the deposited portion solution, suspension or mixture which is different than the color of the tablet core. This can also be measured through other spectroscopic methods such as FTIR or Raman spectroscopy. The diffusion of the deposited portion may be less than 30 mm², or less than 20 mm², or less than 10 mm². The diffusion of the deposited portion may also be measured by calculating the surface area for which the deposited portion has diffused into the tablet as a percentage of the total surface area of the tablet. The diffusion may be less than 20 percent or less than 10 percent of the surface area of the tablet.

In some cases the diffusion is greater when using a coated tablet versus an uncoated tablet, wherein the diffusion is more than 5 percent greater when dispensing on an uncoated tablet versus a tablet that comprises at least 0.1 percent of a film coating by weight of the core.

Another measure of the deposition is the amount of cross-sectional surface area of the tablet that is occupied by the deposited portion. The percentage of area of the tablet may be at least 5 percent, or at least 10 percent, or at least 20 percent of the surface.

Another measure of the deposited portion is that of tablet swelling. Tablet swelling is measured by the percentage of thickness which is increased by the addition of the deposited portion. In the current invention the level of swelling is less than 10 percent, or less than 5 percent.

Multiple deposition steps may be performed within each cavity to create the deposited portion(s). The deposited portion may be created through one to ten deposition steps, or between one and five deposition steps, or between one and three deposition steps.

The tablet comprising deposited portions of the invention can provide a mechanism by which consumers are provided with criteria that are relevant to appropriate selection or deselection of a given product. For example, the tablet comprising deposited portions is presented to the consumer and the consumer simply visually observes decision criteria and selects or deselects a product based on the criteria. Any type of design which functions as a cue as described herein is encompassed by the instant invention.

The “criteria” will have relevance to the decision-making process for deciding whether or not a product is appropriate for, and therefore could be purchased and used by, a consumer considering using the product. Since different criteria for use will apply to different products, the criteria will vary depending on the product being marketed. Examples of criteria include but are not limited to drug, location of symptoms, symptoms treated, time of day for use, drowsy/non-drowsy, form, flavor and combinations thereof.

Criteria as used herein includes both single (i.e., criterion) and multiple (i.e., criteria) characteristics on which a decision may be based. Therefore, criteria may include single or multiple characteristics which are relevant to the decision making process.

Each of the selectable responses will be either positively associated with appropriate purchase and use of the product by a consumer (i.e., a positive selectable response), or negatively associated with appropriate use (i.e., a negative selectable response) and therefore would be associated with deselection of the product.

The term “selection indicia” is intended to mean any observable symbol which is either positively associated with appropriate purchase and use of the product, i.e., positive selection indicia, or negatively associated with appropriate purchase and use of the product, i.e., negative selection indicia. Selection indicia include observable symbols such as graphic symbols including color coding, alphanumeric graphics, pictorial graphics and the like, and sounds such as musical notes, bells, audible language and the like, and combinations thereof. The selection indicia are chosen to be compatible with the design of the dosage form.

For the sake of brevity, the term “indicia” as used herein includes both single symbols (i.e., indicium), such as a single color or graphic, and combinations of symbols (i.e., indicia), such as stripes of alternating colors or a specific color background with a pictorial and/or alphanumeric graphic in the foreground, and the like. Therefore, a single selection indicia may be comprised of one symbol or a combination of symbols which, when observed together as a whole, serve as a single positive or negative selection indicia.

The dosage form of the present invention may be a multilayer tablet, e.g., a trilayer tablet or a bilayer tablet. A bilayer tablet may comprise a modified or sustained release layer and an immediate release layer. One surface of a first layer of a bilayer tablet may comprise the cavities and deposited portion(s) and one surface of a second layer of a bilayer tablet may comprise the alignment feature.

The deposited portion may be comprised of a material that is melted and solidifies upon application of the deposited portion. The deposited portion may cool and harden at room temperature or upon cooling at a temperature less than 25° C. Suitable low-melting hydrophobic materials include polymers, thermoplastic carbohydrates, fats, fatty acid esters, phospholipids, and waxes. Examples of suitable fats include hydrogenated vegetable oils such as for example cocoa butter, hydrogenated palm kernel oil, hydrogenated cottonseed oil, hydrogenated sunflower oil, and hydrogenated soybean oil; and free fatty acids and their salts. Examples of suitable fatty acid esters include sucrose fatty acid esters, mono, di, and triglycerides, glyceryl behenate, glyceryl palmitostearate, glyceryl monostearate, glyceryl tristearate, glyceryl trilaurylate, glyceryl myristate, GLYCOWAX-932, lauroyl macrogol-32 glycerides, and stearoyl macrogol-32 glycerides. Examples of suitable phospholipids include phosphotidyl choline, phosphotidyl serene, phosphotidyl enositol, and phosphotidic acid. Examples of suitable waxes include carnauba wax, spermaceti wax, beeswax, candelilla wax, shellac wax, microcrystalline wax, and paraffin wax; fat-containing mixtures such as chocolate; and the like.

Suitable meltable polymers include hydroxypropyl methylcellulose, hydroxypropyl cellulose, hydroxypropyl methylcellulose acetate succinate, cellulose acetate, ethyl cellulose, polyacrylic acid, polyvinyl alcohol, polyvinylpyrrolidone, pluronics, poloxamers, polyethylene oxide, polyvinyl acetate, polylactic acid and polycaprolactone, and copolymers thereof.

Some active ingredients may be only partially soluble in water and are better suited to deposition in a melt or solvent based deposition system. Such suitable active ingredients include but are not limited to doxylamine succinate, dextromethorphan hydrobromide and chlorpheniramine maleate.

The deposited portion may contain a carbohydrate which melts and flows below 200° C., preferably below 150° C., e.g., “meltable”. Suitable meltable carbohydrates include polysaccharides such as polyfructose, polydextrose, inulin, hydrogen starch hydrosylate, isomalt or polyols such as sugar alcohols including xylitol, sorbitol, erythritol and mixtures thereof.

The deposited portion may be applied as a solvent based solution, and the solvent is subsequently dried off after application to the dosage form. The solvent may comprise ethanol, methanol, hexane, cyclohexane, isopropyl alcohol, dichloromethane, acetonitrile, tetrahydrofuran or acetone. The solution may comprise a hydro-alcoholic system, combining alcohol with water. The solution can also comprise the polymer, carbohydrate, plasticizer, wax, active ingredient and mixtures thereof. Suitable plasticizers for the deposited portion are similar to the materials described above as suitable plasticizers for coating the core.

The deposited portion may comprise a gelling material, or a material that solidifies into a gel upon deposition. The deposited portion may comprise a crosslinked hydrogel material. The dosage form is exposed to visible and/or ultraviolet light after deposition of the appropriate photocurable formulation. The solution can comprise of a photoinitiator, solvent, inhibitors, photocurable oligomer or monomer, light absorber and mixtures thereof. The dosage form is then dried after deposition to remove the water, solvent or combination of both. Suitable gelling materials may include gelatin, pectin, gellan gum, carrageenan, and xanthan gum.

The deposited portion may disintegrate at a different rate than other deposited portions or the core. Where the deposited portion is immediate release, the portion may disintegrate in less than 60 seconds, or less than 30 seconds, or less than 10 seconds. Disintegration testing may be performed using the apparatus and method described in General Chapter 701 of the United States Pharmacopoeia, more specifically the edition USP 43-NF 38.

The deposited portion may comprise a polymer suitable for immediate release of the active ingredient. Polymers suitable for immediate release include but are not limited to water soluble film forming polymers. Suitable water soluble film forming polymers include but are not limited to poloxamers, polyvinyl alcohol, hydroxypropyl cellulose, hypromellose, methylcellulose, pullulan, modified starches, and hydroxyethylcellulose. Polymers suitable for immediate release also include polyols.

The deposited portion may comprise a polymer suitable for pH dependent release of the active ingredient. Polymers suitable for pH dependent release include reverse enteric and enteric polymers. Suitable enteric polymers include hydroxypropylmethylcellulose accetate succinate, cellulose acetate phthalate, hypromellose phthalate, and methcarylic acid-methyl methacrylate copolymers such as those sold under the tradename of Eudragit® L00. Suitable reverse enteric polymers include Amino Methacrylate copolymers such as those sold under the tradename of Eudragit® EPO and E100.

### Process

One preferred process of manufacturing intermediate dosage form begins by compressing or compacting a tablet core into the desired shape of the medicament. As used herein, “compact, compacting, or compacted” and “compress, compressing, or compressed” may be used interchangeably to describe the commonly used process of compacting powders into tablets via conventional pharmaceutical tableting technology as well known in the art. One typical such process employs a rotary tablet machine, often referred to as a “press” or “compression machine”, to compact the powders into tablets between upper and lower punches in a shaped die. This process produces a core having two opposed faces, formed by contact with an upper and lower punch, and having a belly band formed by contact with a die wall. Typically such compressed tablets will have at least one dimension of the major faces at least as long as the height of the belly band area between the major faces. Alternately, processes have been disclosed in the prior art to enable the “longitudinal compression” of tablet cores. When longitudinally compressed tablets are employed, it has been found that an aspect ratio (height between the major faces to width or diameter of the major faces) from about 1.5 to about 3.5, e.g., about 1.9 facilitates handling.

Other processes for producing the core may include confectionary processes such as those typically used for gums and lozenges, such as roping and cutting or molding.

Tablets are typically compacted to a target weight and “hardness”. Hardness is a term used in the art to describe the diametrical breaking strength as measured by conventional pharmaceutical hardness testing equipment, such as a Schleuniger Hardness Tester. In order to compare values across differently sized tablets, the breaking strength is normalized for the area of the break (which may be approximated as tablet diameter times thickness). This normalized value, expressed in kp/cm2, is sometimes referred in the art as “tablet tensile strength.” A general discussion of tablet hardness testing is found in Leiberman et al., Pharmaceutical Dosage Forms—Tablets, Volume 2, 2nd ed., Marcel Dekker Inc., 1990, pp. 213-217, 327-329, which is incorporated by reference herein.

The medicaments manufactured according to the present invention, therefore, provide the desired shape, swallowability and appearance for a solid dosage form. Further, the dosage form of the invention provides improved onset of dissolution and disintegration, while not compromising swallowability of the dosage form. Use of the dosage form in accordance with the invention permits the ability to add actives, colors, flavors, sensates and textures; impart improved swallowability, perception of speed, taste masking, and visual recognition to aid in product selection.

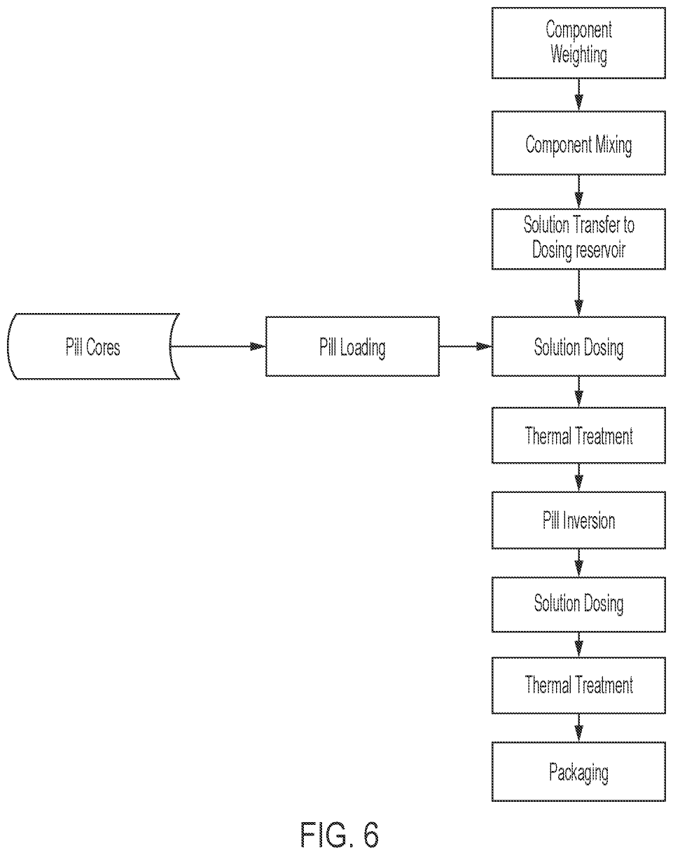

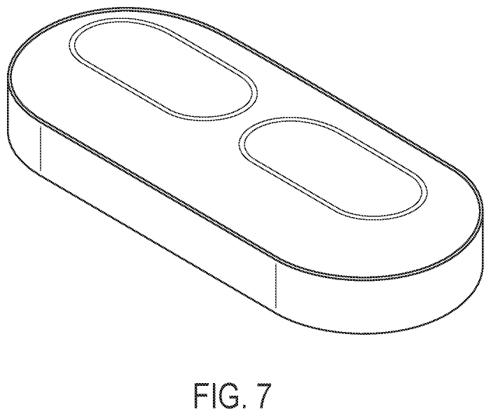

As depicted in FIG. 6, the process of the invention may produce a tablet that comprises cavities and/or deposited portions on two sides or faces of the tablet, such as on the bottom and top of the tablet. The tablet may have the amount or different amounts of cavities and/or deposited portions on the top and bottom of the tablet. In the process, the tablet may be handled such that the deposited portions stay in place or do not migrate between deposition on each face. This may be accomplished by a first deposition on one face; and the addition of a cooling, solidification or drying step and then a second deposition on the second face. This may also be accomplished by orienting or through a captive or positionally controlled rotation of the tablet so that portions can be deposited on the second face. In some examples a combination of (1) a first deposition on one face of the tablet; (2) cooling, solidification and/or drying of the first deposited portion(s), (3) positionally controlled rotation of the tablet, (4) a second deposition on a second face and (5) cooling, solidification and/or drying of the second deposited portion(s) are utilized. This deposition process can be repeated to build the deposited layers to increase the amount of active ingredients or combine different active ingredients within different layers.

The process of the invention may also produce a tablet that comprises cavities on one side or face of the tablet, such as on the top of the tablet, and an alignment feature and/or an identification feature on the opposite side of the tablet, such as on the bottom of the tablet. A protruding alignment feature or debossed alignment feature on the tablet would become attached to a separate indentation or protrusion on an alignment apparatus prior to deposition. Suitable apparatuses for aligning the tablet(s) prior to deposition include feeder bowls, vibratory trays, and rotating brushes. Other alignment features include stamped or printed portions containing an ink, colorant, metal or other chemical which could be identified by a video, visual or spectroscopic inspection system.

The process of the invention may also produce a tablet that comprises cavities on one side or face of the tablet, such as on the top of the tablet and an alignment feature such as a tapered shape or notch at one edge of the tablet.

The deposited portion may be added by a variety of methods. These methods include solution depositing, suspension depositing, melt depositing, ink jet printing, 2D printing or 3D printing. In the case of depositing, the flowable material will be metered out using a specialized pump and a nozzle or printing head. The deposition solution may be maintained in a reservoir that feeds either single or multiple channel pumps. The deposition solution is metered through a nozzle through either volumetric or gravimetric displacement within the pump. The combination of deposition pump displacement distance, speed, and nozzle size defines the volume of deposition solution deposited into the cavity. Variable deposition volumes can be accomplished in situ through changes to the pump displacement distance and speed. The pump displacement is a combination of forward and reverse positioning within a single deposition to mitigate potential impacts associated with droplet size and surface tension within the process. In this manner, the droplet volume, and associated deposition volume, can be controlled outside the constraints of deposition solution surface tension.

In instances wherein the flowable material is deposited on several sides of the dosage form, the dosage form or tablet must be positioned and oriented. Methods for positioning and inspection include vision monitoring systems and controls. Methods for orienting include but are not limited to captive carrier trays, pucks transported on a conveying belt or through the use of robotic transfer through a deposition zone.

The dosage form may be moved or oriented in between deposition steps to accommodate deposition into different cavities, deposition of various compositions into separate cavities or deposition of various compositions into the same cavity. The cavities may be positioned in a line along a surface of a dosage form; e.g. at least two cavities positioned longitudinally along the face of a tablet.

In instances wherein the flowable portion is deposited as a solution or suspension, water or solvent may require removal through the use of a drying step. Suitable drying steps may include infrared heat, convection drying, radio-frequency heating, or microwave heating.

It will become apparent to those skilled in the art that various modifications to the examples of the invention can be made by those skilled in the art without departing from the spirit or scope of the invention as defined by the appended claims.

### EXAMPLES

### Example 1: Preparation of Solution for Depositing

### Solution Preparation:

### Formulation A:

The depositing solution was prepared as follows:
1. Approximately 30 g batches were prepared according to the solution formula in Table 1.
2. DI Water was added to a suitable vessel.
3. Poloxamers, Colorant and Polysorbate were added while mixing, and mixed until dissolved
4. Polyethylene Oxide was added while mixing and mixed until dissolved.

In these experiments, the colorant was a proxy for an active ingredient, which is solubilized in a depositing solution.

TABLE 1
Solution Formulation A

| Ingredients | % .W/W |
|---|---|
| Poloxamer (Kolliphor^{®} P407¹) | 8.00 |
| Poloxamer 188 (Kolliphor^{®} P188²) | 17.00 |
| Colorant (Red 40) | 0.01 |
| Polysorbate-20 | 1.00 |
| Polyethylene Oxide (average mw = 100,000) | 1.00 |
| DI Water | 72.99 |
| TOTAL | 100.0 |

¹Commercially available from BASF Corporation

### Formulations B, C, D:

The depositing solution was prepared as follows:
1. Approximately 30 g batch were prepared according to the solution formula in Table(s) 2, 3 or 4.
2. DI Water was added to a suitable vessel.
3. Poloxamer; Colorant, N-Acetylglucosamine or Niacinamide were added while mixing, and mixed until dissolved.

TABLE 2
Solution Formulation B

| Ingredients | % .W/W |
|---|---|
| Poloxamer (Kolliphor^{®} P407¹) | 12.00 |
| Colorant (Red40) | 8.00 |
| DI Water | 80.00 |

TABLE 3
Solution Formulation C

| Ingredients | %. W/W |
|---|---|
| Poloxamer (Kolliphor ® P407¹) | 12.00 |
| N-Acetylglucosamine (NAG) | 8.00 |
| DI Water | 80.00 |

TABLE 4
Solution Formulation D

| Ingredients | %. W/W |
|---|---|
| Poloxamer (Kolliphor ® P407¹) | 12.00 |
| Niacinamide | 8.00 |
| DI Water | 80.00 |

### Example 2: Core Formulation and Deposition

### Part A: Placebo Core Tablets Prepared by Blending Lactose and Microcrystalline Cellulose

Compressed placebo tablet cores were prepared with Lactose and Avicel PH102, Microcrystalline Cellulose blend with Opadry White 03U180000 film-coating.
The tablets comprise: 84.5% Lactose, 15% Avicel PH102, and 0.5% Magnesium Stearate). The tablet weight was 850 mg.
All of the subsequent examples use the placebo core of Example 2.

### Part B: Deposition Step

The cores from Part A were deposited using the solutions in Example 1. Each solution was deposited using 2 passes of 5.5 μL each. Deposition was completed with an IVEK 40 Pitch linear actuator and 3 A pump with a DS3020 controller. The nozzle was a 20GA blunt needle. The pills were kept on an angled platform and dispensed on one side only. Dispensing was done on 2 cavities/pill at a time using a custom script linked to the translation head of a Jetlab 4 printer from Microfab Technologies, Inc. The parameters used for the IVEK pump can be seen in Table 5.

TABLE 5
IVEK Pump Parameters

| Parameter | Setpoint |
|---|---|
| Direction | Forward |
| Dispense Volume | S1-8 μL / S2-8.2 μL |
| Dispense Meter rate | 50 μL/s |
| Load rate | 50 μL/s |
| Load Threshold | 50 μL |
| Drawback | Disabled |

### Part C: Friability Testing

Formulation A: Deposition: 12% Kolliphor P407
No visual evidence of change at 10 minutes (USP standard friability test).
Minimal visual tablet to tablet migration of deposited material after 30 minutes.
One tablet exhibited breakage across cavity bridge at 90 minutes.

Formulation B: Deposition: 8% Kolliphor P407, 17% Kolliphor P188
No visual evidence of change at 10 minutes (USP standard friability test).
Minimal visual tablet to tablet migration of deposited material after 210 minutes.
One tablet exhibited breakage across cavity bridge at 120 minutes.

### Part D: Tablet Spreading Test

The solutions from Example 1 were deposited into the core cavities and tested for spread efficiency across the area of the cavity. Using a Keyence VR-3200 microscope, the volume and area for the deposited portion was measured using optical software. Both samples (Formulation A and B) had a spreading of greater than 70% of the area of the cavity.

### Part E: Diffusion Test

The solutions from Example 1 were deposited into the core cavities and tested for diffusion into the core as a function of the distance to which the solution diffused below the surface of the cavity. Each deposited sample was cut across the across the caplet circumference (cross section) in order to examine the diffusion. Using a Keyence VR-3200 microscope, the deposition distance was measured using optical software. Both samples (Formulation A and B) had a diffusion of less than 9.5 mm2.

### Part F: Viscosity of Deposition Solutions

Dynamic viscosity was measured on Brookfield viscometer, Model DV3TRVCJ0 using spindle, no: 40z at 25° C. Different shear rates were used depending on the viscosity of the sample being measured. Within the range of shear rates that allowed for the viscosity to be measured (between 10-100% torque range), multiple shear rates were used to sample viscosity and the mean is reflected in Table 6.

TABLE 6
Solution Viscosity

| Formulation | Viscosity (cP) |
|---|---|
| A | 61 |
| B | 122 |
| C | 245 |
| D | 87 |

### Example 3: Preparation of Solution Using Pseudoephedrine

### Formulation E:

The depositing solution is prepared as follows:
1. Approximately 30 g batches are prepared according to the solution formula in Table 7.
2. DI Water is added to a suitable vessel.
3. Pseudoephedrine HCl is added to the water while missing, and mixed until dissolved.
4. Poloxamers and Polysorbate are added while mixing, and mixed until dissolved.

TABLE 7
Solution Formulation E

| Ingredients | %. W/W |
|---|---|
| Poloxamer (Kolliphor ® P407¹) | 15.00 |
| Pseudoephedrine HCl | 20.00 |
| Polysorbate-20 | 1.00 |
| DI Water | 64.00 |
| TOTAL | 100.0 |

¹Commercially available from BASF Corporation

### Example 4: Preparation of Solution Using Phenylephrine HCl

### Formulation F:

The depositing solution is prepared as follows:
1. Approximately 30 g batches are prepared according to the solution formula in Table 8.
2. DI Water is added to a suitable vessel.
3. Phenylephrine HCl is added to the water while missing, and mixed until dissolved.
4. Poloxamer and Polysorbate are added while mixing, and mixed until dissolved.

TABLE 8
Solution Formulation F

| Ingredients | %. W/W |
|---|---|
| Poloxamer (Kolliphor ® P407¹) | 15.00 |
| Phenylephrine HCl | 20.00 |
| Polysorbate-20 | 1.00 |
| DI Water | 64.00 |
| TOTAL | 100.0 |

¹Commercially available from BASF Corporation

### Example 5: Preparation of Solution Using Diphenhydramine HCl

### Formulation G:

The depositing solution is prepared as follows:
1. Approximately 30 g batches are prepared according to the solution formula in Table 9.
2. DI Water is added to a suitable vessel.
3. Diphenhydramine HCl is added to the water while missing, and mixed until dissolved.
4. Poloxamer and Polysorbate are added while mixing, and mixed until dissolved.

TABLE 9
Solution Formulation G

| Ingredients | %. W/W |
|---|---|
| Poloxamer (Kolliphor ® P407¹) | 15.00 |
| Diphenhydramine HCl | 20.00 |
| Polysorbate-20 | 1.00 |
| DI Water | 64.00 |
| TOTAL | 100.0 |

¹Commercially available from BASF Corporation

### Example 6: Preparation of Solution Using Dextromethorphan HBr

### Formulation H:

The depositing solution is prepared as follows:
1. Approximately 30 g batches are prepared according to the solution formula in Table 10.
2. DI Water is added to a suitable vessel.
3. Dextromethorphan HBr is added to the water while missing, and mixed until dissolved.
4. Poloxamer and Polysorbate are added while mixing, and mixed until dissolved.

TABLE 10
Solution Formulation H

| Ingredients | %. W/W |
|---|---|
| Poloxamer (Kolliphor ® P407¹) | 15.00 |
| Dextromethorphan HBr | 20.00 |
| Polysorbate-20 | 1.00 |
| DI Water | 64.00 |
| TOTAL | 100.0 |

¹Commercially available from BASF Corporation

### Example 7: Preparation of Solution Using Chlorpheniramine Maleate

### Formulation I:

The depositing solution is prepared as follows:
1. Approximately 30 g batches are prepared according to the solution formula in Table 11.
2. DI Water is added to a suitable vessel.
3. Chlorpheniramine Maleate is added to the water while missing, and mixed until dissolved.
4. Poloxamer and Polysorbate are added while mixing, and mixed until dissolved.

TABLE 11
Solution Formulation I

| Ingredients | %. W/W |
|---|---|
| Poloxamer (Kolliphor ® P407¹) | 15.00 |
| Chlorpheniramine Maleate | 20.00 |
| Polysorbate-20 | 1.00 |
| DI Water | 64.00 |
| TOTAL | 100.0 |

¹Commercially available from BASF Corporation

### Example 8: Preparation of Polymer Solution Using Chlorpheniramine Maleate

### Formulation J:

The depositing solution is prepared as follows:
1. Approximately 30 g batches are prepared according to the solution formula in Table 12.
2. DI Water is added to a suitable vessel.
3. Chlorpheniramine Maleate is added to the water while missing, and mixed until dissolved.
4. Kollicoat IR® is added while mixing, and mixed until dissolved.

TABLE 12
Solution Formulation J

| Ingredients | %. W/W |
|---|---|
| Macrogol-poly(vinyl alcohol) graft-copolymer, | 10.00 |
| Polyvinyl alcohol-polyethylene glycol graft- | |
| copolymer (Kollicoat IR ® ¹) | |
| Chlorpheniramine Maleate | 30.00 |
| DI Water | 60.00 |
| TOTAL | 100.0 |

¹ Commercially available from BASF Corporation

### Example 9: Preparation of Ethanol Solution Using Loperamide Hydrochloride

### Formulation K:

The depositing solution is prepared as follows:
1. Using non-placebo tablets, specifically tablets with simethicone formulated into the core.
2. Approximately 30 g batches are prepared according to the solution formula in Table 13.
3. Ethanol (95%) is added to a suitable vessel.
4. Loperamide hydrochloride is added to the ethanol (95%) while mixing, and mixed until dissolved.
5. Poloxamer and Polysorbate are added while mixing, and mixed until dissolved.

TABLE 13
Solution Formulation K

| Ingredients | %. W/W |
|---|---|
| Poloxamer (Kolliphor ® P407¹) | 15.00 |
| Loperamide hydrochloride | 4.00 |
| Polysorbate-20 | 1.00 |
| Ethanol (95%) | 80.00 |
| TOTAL | 100.0 |

¹Commercially available from BASF Corporation

### Example 10: Preparation of Solvent Based Formulations

### Formulation(s) L and M:

The depositing solution(s) were prepared as follows:
1. Approximately 30 g batches were prepared according to the formulations listed in Table 14 and 15.
2. Components were dry blended before addition of acetone.
3. Mixing was completed until all components were dissolved.

TABLE 14
Solution Formulation L

| Ingredients | %. W/W |
|---|---|
| Poloxamer (Kolliphor ® P407¹) | 20.00 |
| Ferulic Acid | 30.00 |
| Polyethylene Oxide | 1.00 |
| Polysorbate-20 | 1.00 |
| Colorant (Red40) | 0.01 |
| Acetone | 47.99 |

¹Commercially available from BASF Corporation

TABLE 15
Solution Formulation M:

| Ingredients | %. W/W |
|---|---|
| Poloxamer (Kolliphor ® P188¹) | 20.00 |
| Niacinamide | 30.00 |
| Methanol | 50.00 |

¹Commercially available from BASF Corporation

### Dosing:

Dosing was completed in 2 mL increments for a total of 7 doses to fill the 10 mg target cavity for Formulations L and M at ambient conditions. The solution was allowed to evaporate for 5 minutes between each dose. Thermal treatment after the deposition process was not completed before testing.

### Example 11: Preparation of Aqueous Based Solutions

### Formulation(s) N, O, P:

The depositing solution is prepared as follows:
1. Approximately 30 g batches were prepared according to the formulations listed in the tables 16, 17 and 18.
2. Components were dry blended before addition of water.
3. Mixing was completed until all components were dissolved.

TABLE 16
Solution Formula N

| Ingredients | %. W/W |
|---|---|
| Poloxamer (Kolliphor ® P407¹) | 15.00 |
| Kollicoat ® Protect¹ | 10.00 |
| Niacinamide | 14.00 |
| Polyethylene Oxide | 1.00 |
| Polysorbate-20 | 1.00 |
| Colorant (Red40) | 0.01 |
| Deionized Water | 58.99 |

¹Commercially available from BASF Corporation

TABLE 17
Solution Formula O

| Ingredients | %. W/W |
|---|---|
| Poloxamer (Kolliphor ® P407¹) | 25.00 |
| Niacinamide | 25.00 |
| Polyethylene Oxide | 1.00 |
| Polysorbate-20 | 1.00 |
| Colorant (Red40) | 0.01 |
| Deionized Water | 47.99 |

¹Commercially available from BASF Corporation

TABLE 18
Solution Formula P

| Ingredients | %. W/W |
|---|---|
| Poloxamer (Kolliphor ® P407¹) | 20.00 |
| Niacinamide | 30.00 |
| Polyethylene Oxide | 1.00 |
| Polysorbate-20 | 1.00 |
| Colorant (Red40) | 0.01 |
| Deionized Water | 47.99 |

¹Commercially available from BASF Corporation

### Dosing:

Multiple combinations of dosing were trialed with successful depositions. The following were trialed with Formulations N, O, P. All dose volumes are in mL. Up to 4 cavities were deposited with the dose solutions.

TABLE 19
Dosing Sequences

| Dose | Sequence 1 | Sequence 2 | Sequence 3 |
|---|---|---|---|
| 1 | 12 | 6 | 2 |
| 2 | — | 6 | 2 |
| 3 | — | 3 | 2 |
| 4 | — | — | 2 |
| 5 | — | — | 2 |
| 6 | — | — | 2 |
| 7 | — | — | 2 |
| Total | 12 | 15 | 14 |

After each dose, a thermal treatment process consisting of heating each tablet to 70° C. for 120 seconds. All testing was completed after the final thermal treatment process in each dose sequence.

### Example 12: Preparation of Emulsion Based Solutions

### Formulation(s) Q, R

The depositing solution is prepared as follows:
1. Approximately 30 g batches were prepared according to the formulations listed in table 20 and 21.
2. Components were dry blended before addition of water
3. Mixing was completed until all components were dispersed.

TABLE 20
Solution Formula Q

| Ingredients | %.W/W |
|---|---|
| Poloxamer (Kolliphor ® P407¹) | 11.11 |
| Hyaluronic acid (Hyacare ®-50³) | 6.22 |
| Polyethylene Oxide | 0.44 |
| Polysorbate-20 | 0.44 |
| Colorant (Red40) | 0.01 |
| Deionized Water | 81.76 |

¹Commercially available from BASF Corporation
³Commercially available from the Evonik corporation

TABLE 21
Solution Formulation R

| Ingredients | %. W/W |
|---|---|
| Poloxamer (Kolliphor ® P407¹) | 15.00 |
| Ferulic Acid | 15.00 |
| Polyethylene Oxide | 1.00 |
| Polysorbate-20 | 35.00 |
| Colorant (Red40) | 0.01 |
| Deionized Water | 33.99 |

¹Commercially available from BASF Corporation

### Dosing:

Dosing was completed in 2 mL increments for a total of 7 doses to fill the 10 mg target cavity for Formulations Q and R at ambient conditions. The solution(s) were kept under constant agitation during dispensing to minimize sedimentation. After each dose, a thermal treatment process consisting of heating each tablet to 70° C. for 120 seconds. All testing was completed after the final thermal treatment process in each dose sequence.

### Example 13: Aqueous Solutions with Active Pharmaceutical Ingredients

### Formulation S

The depositing solution was prepared as follows:
1. Approximately 30 g batches were prepared according to the formulations listed in the table 22.
2. Components were dry blended before addition of water.
3. Mixing was completed until all components were dissolved.

TABLE 22
Solution Formula S

| Ingredients | %. W/W |
|---|---|
| Poloxamer (Kolliphor ® P407¹) | 16.7 |
| Active Pharmaceutical Ingredient (API) | 33.3 |
| Polyethylene Oxide | 1.0 |
| Polysorbate 20 | 1.0 |
| Colorant (Red40) | 0.01 |
| Deionized Water | 48.0 |

¹Commercially available from BASF Corporation

Separate solutions were prepared with chlorpheniramine maleate, diphenhydramine hydrochloride, and phenylephrine hydrochloride as the active pharmaceutical ingredient.

### Dosing:

Dosing was completed using the parameters described in Example 11.

### Part A: Diffusion Testing for Aqueous Solutions Using Active Pharmaceutical Ingredients

The samples from Example 13 were tested for diffusion of the solution into the tablet as a function of area diffusion. Cross sections of each tablet were analyzed for diffusion of the solution. The data is shown in Table 22A and averaged for 2 sides of each tablet.

TABLE 22A
Diffusion Results

| | | Diffused Ink | |
|---|---|---|---|
| | Tablet Surface | Surface Area | % |
| Sample | Area (mm²) | (mm2) | Diffused |
| Phenylephrine Tablet Side 1 | 90.44 | ND | N/A |
| Phenylephrine Tablet Side 2 | 92.61 | ND | N/A |
| Phenylephrine Tablet Average | | | N/A |
| Diphenhydramine Tablet Side 1 | 95.14 | 12.65 | 13 |
| Diphenhydramine Tablet Side 2 | 93.98 | 4.44 | 5 |
| Diphenhydramine Tablet Average | | | 9 |
| Chlorpheniramine Tablet Side 1 | 92.30 | 8.93 | 10 |
| Chlorpheniramine Tablet Side 2 | 91.67 | 7.69 | 8 |
| Chlorpheniramine Tablet Average | | | 9 |

ND: None Detected
N/A: Not applicable

### Example 14: Melt Deposition Solutions

### Formulation(s) T, U

The deposition solution(s) were prepared as follows:
1. Approximately 15 g batches were prepared according to the formulations listed in the tables 23 and 24.
2. Pharmaceutical ingredients include: dextromethorphan hydrobromide, chlorpheniramine maleate, diphenhydramine hydrochloride, and phenylephrine hydrochloride.
3. Components were dry blended before heating.
4. Heating was completed based on the melting temperature of the highest melting component plus 20° C.

TABLE 23
Solution for Formula T

| Ingredients | %. W/W |
|---|---|
| Poloxamer (Kolliphor ® P188¹) | 25.00 |
| Pharmaceutical Ingredient² | 75.00 |

¹Commercially available from BASF Corporation
²dextromethorphan hydrobromide, chlorpheniramine maleate, diphenhydramine hydrochloride, and phenylephrine hydrochloride

TABLE 24
Solution for Formula U

| Ingredients | %. W/W |
|---|---|
| Poloxamer (Kolliphor ® P188¹) | 6.70 |
| Polyethylene glycol 200 | 3.30 |
| Pharmaceutical Ingredient² | 90.00 |

¹Commercially available from BASF Corporation
²dextromethorphan hydrobromide, chlorpheniramine maleate, diphenhydramine hydrochloride, and phenylephrine hydrochloride

### Dosing:

Dosing was completed with a linear drive dispensing pump that was maintained at the melt temperatures listed in Table 22. Dose volume target was 10 mL for the 10 mg cavity target. Thermal treatment was not completed before testing.

TABLE 25
Melt Temperatures:

| Pharmaceutical Ingredient | Melt Temperature (° C.) |
|---|---|
| Dextromethorphan hydrobromide | 145 |
| Chlorpheniramine maleate | 153 |
| Diphenhydramine hydrochloride | 186 |
| Phenylephrine hydrochloride | 165 |

### Example 15: Solvent Jetting Formulations

### Formulation(s) V, W, X

The depositing solution(s) were prepared as follows:
1. Approximately 30 g batches were prepared according to the formulations listed in the tables 26, 27 and 28.
2. Components were dry blended before the addition of acetone
3. Mixing was completed until all components were dissolved.

TABLE 26
Solution for Formula V

| Ingredients | %. W/W |
|---|---|
| Poloxamer (Kolliphor ® P407)¹ | 5.00 |
| Ferulic Acid | 5.00 |
| Acetone | 90.00 |

¹Commercially available from BASF Corporation

TABLE 27
Solution for Formula W

| Ingredients | %. W/W |
|---|---|
| hydroxypropyl | 2.00 |
| methylcellulose¹ | |
| Ferulic Acid | 8.00 |
| Acetone | 90.00 |

¹Commercially available as Methocel ™ E5 from the Dupont Corporation

TABLE 28
Solution for Formula X

| Ingredients | %. W/W |
|---|---|
| hydroxypropyl methylcellulose acetate succinate | 2.00 |
| Ferulic Acid | 8.00 |
| Acetone | 90.00 |

### Dosing:

Dosing was completed on a JetLabs 4 system with a MicroFab microdispensing device fitted with a print head with a 35 mm nozzle. Multiple passes were complete to fill the 10 mg cavity target. Thermal treatment was not completed before testing.

### Example 16: Melt Polyol Formulations

### Formulations Y, Z, AA:

### Part A: Preparation and Deposition of One Formula Per Cavity:

The depositing solution was prepared as follows:

Approximately 60 g batches were prepared according to the formula in Table 29, 30, and 31. All components were dry blended on a vortex genie mixer for 30 seconds. The mixture was placed into a heating vessel at 120° C. for 30 minutes before dispensing. Up to 4 cavities were filled.

Part B: Dispensing of Two Formulas into a Single Cavity:

A portion of Formula Y containing Niacinamide from Table 29 (prepared as described in Part A) is first deposited into a single cavity on the core. Subsequently, a portion of the solution from Table 23 containing diphenhydramine hydrochloride is deposited on top of the first formula in the same cavity and allowed to cool to 25° C. In another cavity a portion of Formula Y is first deposited and a portion of the solution from Table 23 containing chlorpheniramine maleate is deposited on top in the same cavity. Up to 4 cavities are filled in this manner, forming a dosage form wherein individual cavities have multiple active ingredients in separate layers.

TABLE 29
Formula Y

| Ingredients | %. W/W |
|---|---|
| Erythritol | 81.00 |
| Xylitol | 9.00 |
| Niacinamide | 10.00 |

TABLE 30
Formula Z

| Ingredients | %. W/W |
|---|---|
| Erythritol | 90.00 |
| Niacinamide | 10.00 |

TABLE 31
Formula AA

| Ingredients | %. W/W |
|---|---|
| Erythritol | 90.00 |
| Xylitol | 10.00 |

### Example 17: Physical Testing

For Examples 10 through 15, the following testing was completed:
Diffusion Test: Diffusion of the dispensed liquid into the cavity was measured
   All tablet cross sections displayed less than 1 mm²
Spreading: The area within the cavity was measured for spread of the dispensed portion.
   All cavities were filled >90% of the area of the cavity.
Cavity volume fill
   All cavities were filled >90% of the possible volume (i.e., 10 mg) without spilling out of the cavity when rotated 30 s after dispensing.
Drop test
   All tablets were dropped from a height of 2 meters at least 10 times without any visible damage to the dosed cavities.

What is claimed is:

1. A dosage form comprising a substrate with two opposing surfaces wherein the first surface comprises at least two cavities and the second opposing surface comprises at least one alignment feature.

2. The dosage form of claim 1, wherein the substrate is a tablet core.

3. The dosage form of claim 1, wherein the at least two cavities on the first surface do not overlap.

4. The dosage form of claim 1, wherein the dosage form comprises two cavities on the first surface.

5. The dosage form of claim 1, wherein the dosage form comprises four cavities on the first surface.

6. The dosage form of claim 1, wherein the at least two cavities on the first surface are separated by at least a 1 mm portion of the surface of the substrate.

7. The dosage form of claim 1, wherein the substrate is elongated.

8. The dosage form of claim 1, wherein the at least two cavities on the first surface are elongated along the same axis as the elongated substrate.

9. The dosage form of claim 1, wherein the at least two cavities on the first surface are equal in size.

10. The dosage form of claim 1, wherein the substrate is coated.

11. The dosage form of claim 1, wherein the at least two cavities on the first surface are capable of receiving up to about 0.05 mL of a flowable material.

12. The dosage form of claim 1, wherein the at least two cavities on the first surface are capable of receiving up to about 50 mg of an active or inactive ingredient.

13. The dosage form of claim 1, wherein at least one of the cavities on the first surface contains an active ingredient.

14. The dosage form of claim 1, wherein at least one of the cavities on the first surface contains an inactive ingredient.

15. The dosage form of claim 1, wherein the substrate contains an active ingredient.

16. The dosage form of claim 1, wherein the substrate contains at least one active ingredient and at least one of the cavities on the first surface contains an active ingredient.

17. The dosage form of claim 1, wherein the substrate contains an active ingredient and at least one of the cavities on the first surface contains an inactive ingredient.

18. The dosage form of claim 1, wherein at least one of the cavities on the first surface contains a visual selected from the group consisting of configuration, color and marking that conveys attributes of the dosage form to a user.

19. The dosage form of claim 1, wherein the at least one alignment feature on the second opposing surface is recessed into the second opposing surface.

20. The dosage form of claim 1, wherein the at least one alignment feature on the second opposing surface is protruding from the second opposing surface.

21. The dosage form of claim 1, wherein the at least one alignment feature on the second opposing surface is a marker printed onto the second opposing surface.

22. The dosage form of claim 1 further comprising an identification feature on the second opposing surface.

23. A method of making a dosage form, comprising:
(a) preparing a substrate with two opposing surfaces and at least two cavities on the first surface and at least one alignment feature on the opposing second surface; and
(b) depositing a flowable material into at least one cavity.

24. The method of making a dosage form according to claim 23, wherein the flowable material covers at least 50% of the bottom surface of the cavity.

25. The method of making a dosage form according to claim 23, wherein the flowable material covers at least 60% of the bottom surface of the cavity.

26. The method of making a dosage form according to claim 23, wherein the flowable material covers at least 70% of the bottom surface of the cavity.

27. The method of making a dosage form according to claim 23, wherein the flowable material diffuses less than 30 mm² into the top surface of the substrate.

28. The method of making a dosage form according to claim 23, wherein the flowable material diffuses less than 20 mm² into the top surface of the substrate.

29. The method of making a dosage form according to claim 23, wherein the flowable material diffuses less than 10 mm² into the top surface of the substrate.

30. The method of making a dosage form according to claim 23, wherein the flowable material comprises a polymer.

31. The method of making a dosage form according to claim 30, wherein the polymer is a poloxamer.

32. The method of making a dosage form according to claim 30, wherein the polymer is selected from hydroxypropyl methylcellulose, hydroxypropyl methylcellulose acetate succinate and polyvinylpyrrolidone.

33. The method of making a dosage form according to claim 30, wherein the polymer is a polyol.

34. A method of making a dosage form, comprising:
(a) preparing a substrate with two opposing surfaces and at least two cavities on the first surface and at least one alignment feature on the second opposing surface;
(b) aligning the substrate using the alignment feature; and
(c) depositing a flowable material into at least one cavity.

35. The method of making a dosage form according the claim 34, wherein the flowable material covers at least 50% of the bottom surface of the cavity.

36. The method of making a dosage form according the claim 34, wherein the flowable material covers at least 60% of the bottom surface of the cavity.

37. The method of making a dosage form according the claim 34, wherein the flowable material covers at least 70% of the bottom surface of the cavity.

38. The method of making a dosage form according to claim 34, wherein the flowable material diffuses less than 30 mm² into the top surface of the substrate.

39. The method of making a dosage form according to claim 34, wherein the flowable material diffuses less than 20 mm² into the top surface of the substrate.

40. The method of making a dosage form according to claim 34, wherein the flowable material diffuses less than 10 mm² into the top surface of the substrate.

41. The method of making a dosage form according to claim 34, wherein the flowable material comprises a polymer.

42. The method of making a dosage form according to claim 41, wherein the polymer is a poloxamer.

43. The method of making a dosage form according to claim 41, wherein the polymer is selected from hydroxypropyl methylcellulose, hydroxypropyl methylcellulose acetate succinate and polyvinylpyrrolidone.

44. The method of making a dosage form according to claim 41, wherein the polymer is a polyol.

45. A method of making a dosage form containing two incompatible active ingredients, comprising:
(a) preparing a substrate with two opposing surfaces and at least two cavities on the first surface and at least one alignment feature on the second opposing surface, wherein the substrate contains an active ingredient;
(b) aligning the substrate using the alignment feature; and
(c) depositing a flowable material containing a second active ingredient into at least one cavity.

46. A method of making a dosage form containing two incompatible ingredients, comprising:
(a) preparing a substrate with two opposing surfaces and at least two cavities on the first surface and at least one alignment feature on the second opposing surface;
(b) aligning the substrate using the alignment feature; and
(c) depositing a first flowable material containing one ingredient into at least one cavity.

47. A method of making a dosage form containing two incompatible active ingredients, comprising:
(a) preparing a substrate with two opposing surfaces and at least two cavities on the first surface and at least one alignment feature on the second opposing surface;
(b) aligning the substrate using the alignment feature;
(c) depositing a flowable material containing an active ingredient into at least one cavity; and
(d) depositing a flowable material containing a second active ingredient into at least one second cavity.

48. A method of making a dosage form containing active ingredients, comprising:
(a) preparing a substrate with two opposing surfaces and at least two cavities on the first surface and at least one alignment feature on the second opposing surface;
(b) aligning the substrate using the alignment feature;
(c) depositing into a cavity a first flowable material containing an active ingredient and a first polymer; and
(d) depositing into the cavity a second flowable material containing an active ingredient and a second polymer.

49. A method of making a dosage form according to claim 48, wherein the first polymer is suitable for pH dependent release of the active ingredient and the second polymer is suitable for immediate release of the active ingredient.

50. A method of making a dosage form, comprising:
(a) preparing a substrate with two opposing surfaces and at least two cavities on the first surface and at least one alignment feature on the second opposing surface; and
(b) depositing a flowable material into at least one cavity.

51. A dosage form comprising a substrate wherein a first surface of the substrate comprises at least two cavities and a second surface of the substrate comprises an alignment feature.

52. A dosage form according to claim 51 wherein the second surface of the substrate is a tapered or notched edge of the dosage form.
